(19) United States
(12) Patent Application Publication
(10) Pub. No.: US 2024/0041788 A1
(43) Pub. Date: Feb. 8, 2024

(54) COMPOSITION COMPRISING A COMBINATION GROWTH FACTORS FOR PREVENTING RADIATION-INDUCED DAMAGE IN CANCER PATIENTS TREATED WITH RADIOTHERAPY

(71) Applicant: UNIVERSIDAD DE LOS ANDES, Las Condes Santiago (CL)

(72) Inventor: Ziyad HAYDAR, Las Condes Santiago (CL)

(21) Appl. No.: 18/258,689

(22) PCT Filed: Dec. 17, 2021
(86) PCT No.: PCT/IB2021/061937

§ 371 Date: Jun. 21, 2023

(30) Foreign Application Priority Data
Dec. 22, 2020 (EP) 20216660.9

(51) Int. Cl. A61K 9/51; A61K 9/127; A61K 38/18; A61P 39/00

(52) U.S. Cl. CPC A61K 9/5161; A61K 9/1271; A61K 38/1808; A61K 38/1825; A61P 39/00

(57) ABSTRACT

The present invention refers to a pharmaceutical composition comprising a release-controlled nanocarrier consisting of core-shell nanoparticles which in turn comprises EGF growth factor and bFGF growth factor. This pharmaceutical composition in used in the context of the present invention for preventing radiation-induced damage in cancer patients treated with radiotherapy.

### FIELD OF THE INVENTION

The present invention refers to the medical field. Particularly, it refers to a pharmaceutical composition comprising a release-controlled nanocarrier consisting of core-shell nanoparticles wherein the nanoparticles comprise EGF growth factor and bFGF growth factor. This pharmaceutical composition in used in the context of the present invention for preventing radiation-induced damage in cancer patients treated with radiotherapy.

### PRIOR ART

Saliva plays a major role in maintaining oral health. This becomes more apparent when the amount and quality of saliva are reduced, often due to medications, Sjögren's syndrome and specially ionizing radiation therapy for tumours of the head and neck, during which the salivary glands are included within the radiation zone. Hyposalivation leading to symptomatic dry mouth or xerostomia, causes difficulty in mastication, swallowing and speech, changes in taste, acceleration of dental caries, oral sores, burning sensations and periodontal diseases. While temporarily alleviated via “intensive” regimens of palliative home and professional care, many patients are unable to maintain the diligence required to be effective.

More considerably, patients affected by salivary gland dysfunction often choose to surrender or terminate their radiotherapy course (and cancer management or treatment) pre-maturely as they become malnourished and experience a significant decrease in their quality of life, mainly due hyposalivation and dry mouth (xerostomia).

So, there is an unmet medical need to find reliable treatments for the prevention of radiation-induced damage, mainly in cancer patients treated with radiotherapy.

The present invention is focused on solving this problem and a specific pharmaceutical composition is herein provided aimed at preventing the radiation-induced damage.

### DESCRIPTION OF THE INVENTION

### Brief Description of the Invention

As explained above, the present invention refers to a pharmaceutical composition (hereinafter the “composition of the invention”) comprising a release-controlled nanocarrier consisting of core-shell nanoparticles wherein the nanoparticles comprise EGF growth factor and bFGF growth factor. This pharmaceutical composition in used in the context of the present invention for preventing radiation-induced damage in cancer patients treated with radiotherapy.

Particularly, the composition of the invention was formulated, characterized and assayed following Examples 1 to 8 of the present application, with particular attention to Example 9 and FIGS. 1 to 10, wherein the positive results obtained with the composition of the invention comprising a specific combination of growth factors included in core-shell nanoparticles are shown. These findings confirm the protective and regenerative effect of the composition of the invention: ((EGF+bFGF)+NPs), over-time, with a favorable capacity to modulate effects via customizing the core-shell format, growth factor load and pharmacokinetic profile.

So, the first embodiment of the present invention refers to a pharmaceutical composition comprising a release-controlled nanocarrier consisting of core-shell nanoparticles wherein the nanoparticles comprise EGF growth factor and bFGF growth factor.

In a preferred embodiment, the nanoparticle core comprises EGF growth factor and the nanoparticle shell is construed via electrostatic-based self-assembly of natural polymers and comprises bFGF growth factor.

In a preferred embodiment, the core-shell nanoparticle comprises a lipid core, and the nanoparticle shell comprises a plurality of layers made of natural polymers.

In a preferred embodiment, the natural polymers are pre-selected blends from: alginates, chitins, chitosans, cellulose-derivates, gelatins and/or hyaluronans.

In a preferred embodiment, the lipid core comprises liposomes or solid lipid nanoparticles.

The second embodiment of the present invention refers to the pharmaceutical composition of the invention for use in preventing radiation-induced damage in cancer patients treated with radiotherapy.

In a preferred embodiment, the present invention refers to the pharmaceutical composition of the invention for use in preventing radiation-induced damage in patients suffering from head and neck cancer who are treated with radiotherapy.

In a preferred embodiment, the present invention refers to the pharmaceutical composition of the invention for use in preventing salivary glands damage and/or dry mouth complications.

In a preferred embodiment, the present invention refers to the pharmaceutical composition of the invention for use in preventing xerostomia, halitosis, burning mouth syndrome or ulcers.

In a preferred embodiment, the present invention refers to the pharmaceutical composition of the invention for use in the reparation, reconstruction or regeneration of the salivary glands.

In a preferred embodiment, the growth factors are continuously delivered for a pre-established period of time, preferably for 30-45 days.

In a preferred embodiment, the growth factors are sequentially delivered in a manner that bFGF is released first and EGF is delayed for a pre-established period of time.

In a preferred embodiment, the growth factors are delivered at a pre-established rate.

In a preferred embodiment, the composition is directly injected into the high-risk/susceptible site, preferably into the salivary glands.

In a preferred embodiment, 100-500 ng/mL are administered 24 hours to 6 hours before radiotherapy session.

The third embodiment of the present invention refers to a method for preventing radiation-induced damage in cancer patients treated with radiotherapy, for preventing radiation-induced damage in patients suffering from head and neck cancer who are treated with radiotherapy, for preventing salivary glands damage and/or dry mouth complications, for preventing xerostomia, halitosis, burning mouth syndrome or ulcers, or for the reparation, reconstruction or regeneration of the salivary glands, which comprise the administration of a therapeutically effective amount of the pharmaceutical composition of the invention. In a preferred embodiment, the pharmaceutical composition of the invention may comprise pharmaceutically acceptable excipients or carriers.

For the purpose of the present invention the following terms are defined:
The term “comprising” means including, but it is not limited to, whatever follows the word “comprising”. Thus, use of the term “comprising” indicates that the listed elements are required or mandatory, but that other elements are optional and may or may not be present.
By “consisting of” means including, and it is limited to, whatever follows the phrase “consisting of”. Thus, the phrase “consisting of” indicates that the listed elements are required or mandatory, and that no other elements may be present.
“Pharmaceutically acceptable excipient or carrier” refers to an excipient that may optionally be included with the composition of the invention and that causes no significant adverse toxicological effects to the patient.
By “therapeutically effective dose or amount” of a composition comprising the peptide of the invention is intended an amount that, when administered as described herein, brings about a positive therapeutic response in a irradiated subject. The exact amount required will vary from subject to subject, depending on the age, and general condition of the subject, the severity of the condition being treated, mode of administration, and the like. An appropriate “effective” amount in any individual case may be determined by one of ordinary skill in the art using routine experimentation, based upon the information provided herein.

### DESCRIPTION OF THE FIGURES

FIG. 1. Representative submandibular glands of a male C57BL/6 mice, analysed histomorphometrically, comparing control and experimental strategies. A) unirradiated (unIR) control-saline injection; B) irradiated (IR) experimental-cocktail injection of the composition of the invention.

FIG. 2. Surface density (Sv) and Length density (Lv) in ducts determined in irradiated (IR) and unirradiated (unIR) models in different conditions. The best results achieved with the composition of the invention are shown.

FIG. 3. Unirradiated (un-IR) glands treated with different conditions. A) un-IR saline; B) un-IR NPs; C) un-IR EGF; D) un-IR EGF+NPs; E) un-IR bFGF; F) un-IR bFGF+NPs; G) un-IR EGF+bFGF; H) un-IR EGF+bFGF+NPs, showing the best results achieved with the composition of the invention.

FIG. 4. Irradiated glands (IR) treated with different conditions. A) IR saline; B) IR NPs; C) IR EGF; D) IR EGF+NPs; E) IR bFGF; F) IR bFGF+NPs; G) IR EGF+bFGF; H) IR EGF+bFGF+NPs, showing the best results achieved with the composition of the invention.

FIG. 5. Radiation damage prevention and damage reparation in a sub-mandibular gland of a male mouse C57BL/6. A) H&E staining B) Picrosirius Red. Tubulo-Alveolar-type Glandular Tissue can be clearly observed. Granular Convoluted Ducts (*) and Intra-Lobular Ducts (+) are surrounded by connective tissue collagen fibers type I (in red or yellow) and type III (in green).

FIG. 6. Apoptotic cells for each treatment.

FIG. 7. Salivary function assessment determined at day 30 (d30) and day 60 (d60) post-IR. It is expressed as ratio of post-IR SFR (salivary flow rate) to pre-IR SFR (mean±standard error), showing the best results achieved with the composition of the invention.

FIG. 8. Detection of apoptosis by tunnel assay in different conditions. Ctrl) UnIR; A) IR saline; B) IR NPs; C) IR EGF; D) IR EGF+NPs; E) IR bFGF; F) IR bFGF+NPs; G) IR EGF+bFGF; H) IR EGF+bFGF+NPs, showing the best results achieved with the composition of the invention.

FIG. 9. PCNA immunohistochemistry. The number of proliferating cells in tissue sections are determined via Proliferating Cell Nuclear Antigen (PCNA) staining (anti-PCNA monoclonal antibody is a marker for proliferating cells—acinar and granular convoluted tubular cells), showing the best results achieved with the composition of the invention.

FIG. 10. PCNA staining. Number of proliferative cells are determined in different conditions. The best results achieved with the composition of the invention are shown.

### DETAILED DESCRIPTION OF THE INVENTION

The present invention is illustrated by means of the Examples set below, without the intention of limiting its scope of protection.

### EXAMPLE 1. DEVELOPMENT OF A DUAL-RELEASE DRUG DELIVERY SYSTEM

In vitro step-wise synthesis for liposomes formulation: Preparation of rhEGF-loaded lipid vesicles (core) and preparation of L-b-L self-assembled polymeric shell loaded with rhbFGF. Nano liposomes are formulated via the thin-film hydration technique, that allows to yield uniform and stable LUVs (large uni-lamellar vesicles). Then, encapsulation will be performed by extrusion and ultracentrifugation. Size and charge characteristics will be determined using HPPS-DLS nanosizer.

In vitro synthesis for dual-drug delivery system: Liposomes are formulated via the thin-film hydration technique to prepare dual-loaded core-shell nanoparticles. A lipid phase is prepared by dissolving 1,2-Dipalmitoyl-sn-glycerol-3-phosphocholine (DPPC), cholesterol (Chol) and di-methyl-dioctadecyl-ammonium bromide (DDAB). DPPC:CHOL:DDAB in 7:2:1 molar ratio. Each lipidic phase was prepared individually in a chloroform-methanol mixture (4:1 v/v). DDAB is used in a 4% molar concentration to tailor the surface charge of the liposomes. The solvent mixture is then removed from the lipid phase by rotary evaporation (SCI-LOGEX re100 PRO, Laboratorio BioMAT'X, Universidad de Los Andes), 60° C., 150 rpm under vacuum resulting in the deposition of a homogenous dry lipid film. The film is hydrated with ultra-pure water (UPW) (60° C.), vortexed to obtain a cloudy suspension of positively-charged multi-lamellar vesicles and transferred into a mini extruder (Avanti mini extruder N610000, Laboratorio BioMAT'X, Universidad de Los Andes), previously heated to 60° C., with two 100 nm pore size 19 mm polycarbonate filters (GE Osmonics), the sample was extruded 21 times to obtain liposomes of homogeneous size (named L). Liposomes suspensions were freeze-dried at −54° C. for 48 hours and stored at −20° C. until use.

For the encapsulation accurately weighed lyophilized LUVs (2 mg) are then re-hydrated to the original volume (2 mL) with a rhEGF (6.2 kDa molecular weight, Life Technologies) solution (300 μg/mL), the factor incorporation is induced with orbital agitation (1100 rpm, 30 min), forming the nanoparticles core, composed of EGF-loaded liposomes (L-EGF). The EGF-loaded liposomes are the separated from the un-absorbed protein by ultracentrifugation (30 min, 180.000×g, 25° C.). Finally, the un-adsorbed EGF in the supernatant is quantified using a colorimetric method (Micro BCA protein assay, Life Technologies) by reading the absorbance at 562 nm (Infinite m200 pro, TECAN). The loading capacity (LC) and encapsulation efficiency (EE) of the LUVs are calculated using the equations below:

$$LC = \frac{{ECF}_{Total} - {EGF}_{Super}}{{Mass}_{Vesicles}} \times 100 \\ Loading Capacity \qquad Equation 1$$

$$EE = \frac{{EGF}_{Total} - {EGF}_{Super}}{{EGF}_{Super}} \times 100 \\ Encapsulation Efficiency \qquad Equation 2$$

where EGF_{Total} is the initial amount of rhEGF; EGF_{Super} is the amount of un-adsorbed rhEGF measured in the supernatant, and Mass_Vesicles is the mass of the initial EGF-loaded vesicles powder lyophilized.

For the layer-by-layer (L-b-L) build-up, 0.5 mg/mL solution of alginate is prepared in UPW and chitosan 1 mg/mL solution is prepared in 1% (v/v) acetic acid aqueous solution and the final pH adjusted with 1M NaOH to 5.5. The cationic EGF-core vesicles are coated with alternating layers of negatively charged alginate (AL) and positively charged chitosan (CH) (volume ratio 1:2 respectively) until 5 bi-layers (10 layers) are achieved. During the construction of the coating, exactly 0.5 mg/mL rhbFGF (17 kDa molecular weight, Life Technologies) solution was divided and step-/drop-wise added to the process (named L EGF (AL-CH)s FGF).

Loading Capacity (LC) and Encapsulation Efficiency (EE): Unabsorbed EGF in the supernatant, after EGF-core formulation, was quantified using Micro BCA protein assay method (Life Technologies) by reading the absorbance at 562 nm (Infinite m200 pro, TECAN), loading capacity (LC) and encapsulation efficiency (EE) of the LUVs were calculated using the equations [symbol] Error! No se encuentra el origen de la referencia. [symbol] Error! No se encuentra el origen de la referencia., considering 600 μg as EGF_{Total} and 2 mg as Mass_{Vesicles}. The results obtained are showed in Table 1.

TABLE 1

| | Loading Capacity | Encapsulation Efficiency |
|---|---|---|
| Total protein (average) | (LC) | (EE) |
| 166.55 ± 15.14 μg/mL | 60.67 ± 20.22 | 84.5 ± 35.17 |

EGF-core loading characterization

Likewise, in a separate experiment, for the constructed 10-layered shell, loaded with 0.5 mg/mL rhbFGF, un-adsorbed bFGF in the supernatant, was quantified using the Micro BCA protein assay method (Life Technologies) by reading the absorbance at 562 nm (Infinite m200 pro, TECAN), loading capacity (LC) and encapsulation efficiency (EE) of the shell were calculated for 500 lag bFGF_{Total} and 0.5 mg as Mass_{Shell}. The results obtained are showed in Table 2.

TABLE 2

| | Loading Capacity | Encapsulation Efficiency |
|---|---|---|
| Total protein (average) | (LC) | (EE) |
| 85.45 ± 27.17 μg/mL | 43.72 ± 33.14 | 64.3 ± 28.32 |

bFGF-shell loading characterization

The formulation loading was optimized by the preparation of 5 different batches. The best formulation showed 0.27 mg of GFs/mg of NPs and 53% of the GFs successfully entrapped in the nanoparticles.

Physico-Chemical parametric study: The physic-chemical parameters average hydrodynamic diameter, polydispersity index (PI, size distribution), surface charge and mean count rate (MCR) were measured during the L-b-L construction in loaded and un-loaded nanoparticles by Zetasizer nano Zs3000 (conducted at CIMIS, UNAB, Santiago de Chile).
Average hydrodynamic diameter (nm): The average hydrodynamic diameter was measured in different stages of the formulation process. In the core-shell formulated nanoparticles, the average hydrodynamic diameter increases as the number of layers increase, however, after adding the last layer of each polymer the diameter decreases due to the strong electrostatic interaction between the polymers causing a compaction (a denser polymer interconnected network), with an associated reduction of the hydrodynamic diameter. Additionally, comparing loaded and unloaded, the first are slightly larger, comparing both nanoparticles with 5 bi-layers each, product of the incorporation of the growth factors. Basically, as expected, based on our previous expertise with the formulation process, the more layers deposited onto the core, the denser and more compact the nanocapsular system gets. This characteristic is favorable, in order to enhance stability, better protect the load, as well as prolong the release pharmacokinetics. In addition, this provides an option of formulation a slower vs faster drug delivery system.
Polydispersity index (PDI): The polydispersity index is an indicator of the homogeneity of the nanoparticles in the sample, this index goes from 0 to 1, and a value ≤0.4 is considered acceptable value of dispersity (homogenously distributed) of the particles to be used in (proceed to) further experiments. Comparing unloaded nanoparticles vs loaded nanoparticles, both have an acceptable PDI 0.333 for un-loaded and 0.227 for loaded nanoparticles, meaning that nanoparticles solutions with components of a homogeneous size were obtained.
Surface charge: The net surface charge of the particle is determined through Z-potential; a critical parameter of stability. This is a very important parameter to consider, since all the interaction of the particles with any cell or tissue starts with specific electrostatic interactions. Furthermore, it has been reported that negative charged nanoparticles are recognized by proteins in the bloodstream and trigger an agglomeration of these proteins when they interact with the nanoparticles. Due to the above, nanoparticles should be slightly positive. The switching in the surface charges is evidence of different polymer deposition. The net surface charge of the 5 bi-layer nanoparticles L EGF (AL-CH)₅ is +20.70 mV, very well within the accepted range of stable nano-suspensions formulated for physiologic injection (±30 mV).
Mean Count Rate (MCR): The mean count rate was determined in different stages of formulation process. The values obtained for the physical-chemical characterization of initial and final stages of the formulation are shown in
Table. The values obtained for hydrodynamic diameter (217.8±19) and Z potential (+20.75±1.6) are within those proposed in the project (250400 nm and 15-40 mV, respectively); and both values are also within the values reported in the literature. If a slight difference is detected in the data, it can be attributed to the fact the nanoparticles formulated in this project are loaded with (very) low molecular weight proteins such as rhEGF (6.2 kDa) and rhbFGF (17 kDa) and the nanoparticles previously reported were loaded with BSA and BMPs.

TABLE 3

| | Hydrodynamic | | MCR | | | Z Potential | |
|---|---|---|---|---|---|---|---|
| Sample | Diameter (nm) | SEM | (kcps) | PDI | SEM | (mV) | SEM |
| L | 84.86 | 8.38 | 280.55 | 0.333 | 0.047 | 42.2 | 2.08 |
| L-EGF | 94.58 | 8.10 | 257 | 0.293 | 0.089 | 44.33 | 5.61 |
| L(AL-CH)₅ | 184 | 33.23 | 255 | 0.333 | 0.010 | 23.18 | 2.63 |
| L-EGF (AL- | 217.76 | 18.75 | 255 | 0.227 | 0.032 | 20.70 | 1.55 |
| CH)₅ FGF | | | | | | | |

Physico-Chemical characterization of initial and final stages of nanoparticles formulation

The different parameters (Hydrodynamic diameter, PDI, Z potential and MCR) were measured after the incorporation of each polymeric layer, for nanoparticles without proteins (unloaded) and with proteins (loaded), results are shown in [symbol] Error! La autoreferencia al marcador no es válida.

### Error! La autoreferencia al marcador no es válida.

| | Hydrodynamic | | | | Z potential | | | |
|---|---|---|---|---|---|---|---|---|
| | Diameter (nm) | | PDI | | (mV) | | MCR | |
| | Unloaded | Loaded | Unloaded | Loaded | Unloaded | Loaded | Unloaded | Loaded |
| L | 84.87 ± | 94.50 ± | 0.33 ± | 0.293 ± | +42 ± | +44.3 ± | 280.6 ± | 283.6 ± |
| | 17 | 16 | 0.08 | 0.18 | 4 | 9.7 | 93 | 84 |
| LA₁ | 234.20 ± | 277.25 ± | 0.429 ± | 0.314 ± | −56.3 ± | −60 ± | 235.4 ± | 213.2 ± |
| | 54 | 75 | 0.15 | 0.21 | 0.6 | 1 | 97 | 95 |
| LA₁CH₁ | 265.4 ± | 275.67 ± | 0.325 ± | 0.351 ± | +36.8 ± | +36 ± | 306.3 ± | 313.1 ± |
| | 43 | 9 | 0.06 | 0.06 | 6.1 | 7.8 | 55 | 69 |
| LA₂CH₁ | 344.3 ± | 202.25 ± | 0.409 ± | 0.444 ± | −44 ± | −43.67 ± | 278.9 ± | 255.4 ± |
| | 83 | 90 | 0.1 | 0.14 | 8.7 | 5 | 105 | 73 |
| LA₂CH₂ | 381.15 ± | 258.25 ± | 0.458 ± | 0.426 ± | +27 ± | +29.75 ± | 285.1 ± | 231.9 ± |
| | 128 | 106 | 0.07 | 0.13 | 3.6 | 5 | 50 | 115 |
| LA₃CH₂ | 346.13 ± | 185 ± | 0.395 ± | 0.404 ± | −42.5 ± | −41 ± | 229.9 ± | 320.6 ± |
| | 90 | 35 | 0.09 | 0.19 | 3.7 | 4.6 | 74 | 71 |
| LA₃CH₃ | 342.53 ± | 189.25 ± | 0.427 ± | 0.527 ± | +25.8 ± | +31 ± | 145.4 ± | 249.6 ± |
| | 86 | 90 | 0.13 | 0.05 | 8.2 | 3.4 | 26 | 84 |
| LA₄CH₃ | 347.28 ± | 287.25 ± | 0.347 ± | 0.461 ± | −41.3 ± | −39.67 ± | 284.9 | 314.8 ± |
| | 178 | 75 | 0.03 | 0.2 | 6.4 | 2.1 | | 23 |
| LA₄CH₄ | 518.9 ± | 223.67 ± | 0.285 ± | 0.428 ± | +26 ± | +28.5 ± | 166.6 ± | 356.4 ± |
| | 245 | 64 | 0.11 | 0.2 | 2.7 | 2.4 | 66 | 9.6 |
| LA₅CH₄ | 292.3 ± | 290 ± | 0.316 ± | 0.403 ± | −34 ± | −37.7 ± | 142 ± | 239.3 ± |
| | 79 | 109 | 11 | 0.15 | 11.6 | 4.7 | 40 | 42 |
| LA₅CH₅ | 184 ± | 217.8 ± | 0.333 ± | 0.227 ± | +22.5 ± | +20.75 ± | 187.9 ± | 255.4 ± |
| | 58 | 19 | 0.02 | 0.06 | 5.2 | 1.6 | 57 | 80 |

Physico- Chemical characterization of the different stages of nanoparticles formulation for nanoparticles with growth factors (loaded) and without growth factors (unloaded). (AL: Alginate, CH: Chitosan, subscript: number of polymeric layers; Data corresponds to mean ± SD).

After synthesis, completion the nanocapsules were lyophilized and stored at −20° C. until use. The results presented in this section correspond to the first milestone of the project, the development of core-shell nanocapsules as a dual-drug delivery system, showing an efficient synthesis of the nanoparticles together with the complete characterization of the physico-chemical parameters, with a hydrodynamic diameter of 217.76±18.75 nm and a Z potential of +20.7±1.55 mV. According to the results presented, the first milestone was successfully completed. It is also possible to lower the size of the nanocapsules when using solid lipid nanoparticles and/or SUVs; a set of experiments we have performed on the side. The formulated nano-suspension is clear and flowable (low viscosity), hence, suitable for clinical syringe-injection. Also, the performed lyophilization process was deemed successful in preserving its characteristics.

The nanoparticles and the principal parameters for its characterization show a homogeneous distribution of sizes and this increase when GFs are loaded, showing that the protein are successfully incorporated into the nanocapsules. The fluctuations on surface charge evidence the foliation of alternating polymeric layers in the surface, proving that the formulation of nanocapsules was achieved.

### EXAMPLE 2. CHARACTERIZATION OF DUAL-RELEASE DRUG DELIVERY SYSTEM

The formulated core-shell nanoparticles were analyzed morphologically by transmission electron microscopy, scanning electron microscopy, atomic force microscopy, and the conformational changes by differential scanning calorimetry from 4-150° C., during the synthesis and lyophilization process.

After the core-shell nanoparticles synthesis and the evaluation of physic-chemical parameters, finding that these last were within the expected values, morphological characteristics and stability were assessed. The samples obtained from the previous formulation were prepared for its analysis by transmission electron microscopy (TEM) in the “Unidad de Microscopia Avanzada UC” facilities of Pontificia Universidad Catolica de Chile (an out-sourced activity).

### Morphological Characterization of Dual-Release Drug Delivery System:

Electron Transmission Microscopy (TEM): Throughout the formulation of core-shell nanoparticles the morphology remains constant, liposomes, liposomes after the incorporation of the first bi-layer (L(AL-CH)1) and the nanoparticles with 5 polymeric bi-layers (L(AL-CH)5) maintain their spherical structure. The size of the nanocapsules is different from what was previously reported since they are affected by the processing associated to the transmission electron microscopy, mainly by the associated vacuum system that is necessary for the operation of the electrons beam that builds the images.
Atomic Force Microscope (AFM): Besides the characterization by TEM, the morphological stability of the nanoparticles was assessed by atomic force microscopy (AFM), surface characteristics were measured for unloaded liposomes and the formulation with 3 bilayers. For both morphological measurements the formulation has a stable spherical structure, allowing to determinate the structural stability of the formulation. The height profile explains the differences in the construction of the shell and the thickness of the polymeric coating and the stability to support the weight and pressure of the probe. Likewise, this parameter probes the ability of the nanoparticles of carrying a load/factor or agent inside or in its shell, which optimized the loading capacity (LC) and encapsulation efficiency (EE) in addition to allow a controlled released in time of the agent loaded in the formulation.

Stability of dual-release drug delivery system: In order to assess the stability of the formulation Differential Scanning calorimetry (DSC) measurements were performed. The assay is performed in aluminum wells with a maximum volume of 100 μL. 15 mg of lyophilized formulation were used in each measurement, and the assay was performed, the assay was performed in a range of temperature from 4 to 150° C. (this range was selected according to aluminum properties) with a ramp of 5° C. per minute, in DSC 1 Star System (Mettler, Toledo). The data was normalized by the mass of the lyophilized formulation in each measurement.

Thermal energy profiles were created, with peaks that represent phase change of lipids in the formulation (around 60° C.), only one peak means that in the lipid formulation exists only one chemical structure. Liposomes without protein (blue line) present a second peak (≈90° C.), this could be explained by the loss of the spherical shape and the composition of the liposome, showing some instability of the formulation. Liposome have an absorption energy of 0.462 mW/mg before the phase change peak, after this the absorption energy rises in 0.003 mW/mg up to 0.465 mW/mg.

When liposomes are loaded with BSA they have an absorption energy of 0.630 mW/mg before the peak and of 0.680 mW/mg after the peak, the 0.050 mW/mg difference can be attributed to encapsulated BSA. Due to BSA is a protein, a phase change can't be evidenced, but when a protein is denatured, losing its secondary and tertiary structure, their absorption energy capacity increases phenomenon that explains the differences in formulation's absorption energy.

Nevertheless, after the incorporation of the polymeric coating to the liposome's thermal energy profiles show only one peak, fact that can be interpreted as an increased stability provided by the polymeric coating. With the analysis of thermal energy profiles, it's possible to conclude that after the incorporation of the polymeric coating, stability increases, as well as, the differences in the absorption of energy before and after the lipid phase change when liposomes and core-shell nanoparticles are loaded with a protein mean that a denaturation process is occurring in the molecule, what demonstrate that the protein molecule, BSA, has been successfully incorporated to the formulation.

The results showed above prove the stabilization of the nanoparticles due to the construction of a polymeric coating (protective shell) and the presence of proteins into the nanoparticle structure.

Interaction of dual-release drug delivery system by fluorescence microscopy: To assess the interaction of the dual-release drug delivery system with cells, nanoparticles were loaded with Quantum Dots (QD). Liposomes were incubated with agitation, 1 h at 37° C. followed by centrifugation (180.000 g, 25° C., 10 min), supernatants was removed to eliminate non-internalized QDs and 10 polymeric layer were constructed on loaded liposomes to obtain L QDs(AL-CH)₅. In parallel, 30.000 NIH/3T3 cells were seeded on a glass cover and incubated for 1 h at 37° C., 5% CO₂, L QDs(AL-CH)₅ solution was added and incubated for 1 h, 37° C., 5% CO₂. After the incubation, cells were fixed, stained with DAPI and watched in fluorescence microscope (60× magnitude). L QDs(AL-CH)₅ nanoparticles stay mostly outside the cell which suggest an extracellular release of growth factors loaded into the core-shell nanoparticles, being able of an activation of growth factor's receptors localized on the outside of the cell membrane. These results support the mechanism of action proposed for this technology and the functionality of core-shell nanoparticle as a drug delivery system for growth factor's delivery. To demonstrate localization and/or internalization of nanoparticles, they will be analyzed with fluorescence microscopy following previously described methodology. Nanoparticles loaded with Quantum Dots (QDs) (CdSe/ZnS core-shell type quantum dots) and a β-actin antibody along with a secondary antibody with fluorescent labelling to evaluate if the formulation is being internalized (Peñaloza, 2017). If nanoparticles are internalized, they will be followed through labeling with LysoTracker probes (Green DND-26) that allows to observe acidification within phagolysosomes in 1 and 4 hours after treatment according to previously reported protocol (Pefialoza, 2017). Together, these experiments will allow to assess intracellular purpose of the formulation.

### EXAMPLE 3. RELEASE KINETICS PROFILES OF DUAL-RELEASE DRUG DELIVERY SYSTEM

Release kinetics show the accumulative total protein concentration in time (days), exists a controlled release until day 5, after that there is a decrease in the released proteins followed of stable release kinetics from day 6 to 9, where the proteins concentration reaches a peak. When growth factors are measured separated rhEGF shows a faster release than rhbFGF, achieving maximum liberation around day 5. On the other hand, rhbFGF presents a slower initial release, increasing from day 5.

The cause of such differences in the release profiles could be attributed to the variance in size of both factors (rhEGF is smaller than rhbFGF), load location (rhEGF in Core and rhbFGF in Shell), and load/release timing (rhEGF diffusing earlier and faster from Core than rhbFGF loaded within the different compartments of the shell; a dense and compact shell—via diffusion and erosion of the interpenetrating polymer network).

Many of mesenchymal cells culture medium contain rhEGF and rhbFGF (5:1), considering this the difference observed are not necessary a problem or complication on the potential radioprotector effect of salivary glands, yet, out of curiosity, this experiment has been repeated for longer time frame (up to 60 days). Complete results of 60 days release profile showed a sequentially sustained linear release of the growth factors up to day 60 (55 days), and not reaching a plateau yet.

### EXAMPLE 4. CYTOCOMPATIBILITY AND BIOACTIVITY OF PRE-CLINICAL PROTOTYPE

The cytocompatibility and bioactivity evaluated in vitro by (a) assessing cell proliferation and viability and (b) quantifying their ability to stimulate DNA synthesis in murine and human fibroblasts culture cells (ATCC CCI163 and PCS-201-010) for 48h, 37° C. For quantification commercial CellQuantiMTT kit is used. For in vivo evaluation C57BL/6 mice aged 7-9 weeks will be injected with different formulations (empty nanocapsules, control or nanocapsules loaded with growth factors), later head and neck region will be locally irradiated. Animals will be checked daily to follow up their general health status.

To assess the bioactivity of nanoparticles first, it's necessary to determine toxicity of the nanoparticles on the cells lines that will be evaluated. These assays were performed with unloaded core-shell nanoparticles (L(AL-CH)₅) on murine fibroblasts NIH/3T3 from the American Type Culture Collection (ATCC) and on human cell lines Cal27 and HEK293 to evaluate the effect of the formulation on the different tissues present in the salivary gland. Cells were growth in DMEM medium, 2 mM glutamine and 10% FBS (37° C., 5% CO₂), to perform the experiments 5.000 cells were seeded in 96-well plates, incubated 24 h and stimulated with the nanoparticles in concentrations from 0.01 μg/mL to 500 μg/mL, after 24 h cell viability was assessed with PrestoBlue kit. As negative control cells were treated with normal culture medium without nanoparticles.

None of the tested nanoparticles concentrations showed a statistically significative difference in cytotoxicity compared to control, statistical analysis was performed through one-way ANOVA (p>0.05). With the obtained data is possible to determinate the bioactivity of loaded core-shell nanoparticles (L EGF (AL-CH)₅ FGF) which are expected to induce cell proliferation.

In order to assess the effect of growth factor on cell cultures, different concentration of growth factor was assessed. The assayed values correspond to those previously reported in literature in a range from 1 to 50 ng of each growth factor to stimulate cell proliferation in vitro on different types of cell lines such as mesenchymal, fibroblasts and epithelial cell lines (Wang, 2015; Lee, 2015; Stoll, 2015). First, epithelial growth factor (rhEGF) and fibroblast growth factor (rhbFGF) on Cal27 cells, cell line that proceeds from an epithelial carcinoma of the tongue. The cultures were growth in DMEM medium, 10% FBS (37° C., 5% CO₂) and subcultured with trypsin/EDTA after reaching 80-90% confluence.

Growth factors stimulation assays were performed in flat bottom 96-wells plates, every condition was evaluated in triplicate seeding 5.000 cells in each well in a final volume of 100 μL of medium, cells were incubated for 24 h until cells attachment, washed with PBS 1× and added fresh culture medium supplemented with growth factors in the corresponding concentrations, after 24 h cell viability was determinate with PrestoBlue reagent according to manufacturer instructions (1:10 in complete medium, 1 h, 37° C., 5% CO₂), this reagent gives a measure of the metabolic activity of cells, value that can be correlated with the amount of living cells in the culture. After incubation absorbance was measured in infinite M200 Pro TECAN (570 nm, reference wavelength), as control cells with normal growing conditions and negative control for cell death were performed with 70% methanol for 30 minutes.

Cell viability on Cal27 cell line: Cell viability tends to increase when Cal27 cells were stimulated with rhEGF but ANOVA test doesn't show statically significative differences. When cells are stimulated with rhbFGF cell viability decreases with concentrations equal or higher than 5 ng, when a growth factors cocktail was applied a tendency of increasing cell number is observed but no statistically significative differences were showed. These results are not the ones expected nor are they related to the reported data, where in concentrations of 5 ng of rhbFGF is possible to observe a 150% cell viability after 24 h.

Although the efforts to obtain results with a better SD, this was not possible, so the experiments were also performed on NIH3T3 and HEK293 cell lines, but results were similar, and they didn't represent the previously reported data.

Looking for the cause of the problem, we observed that all cell cultures were losing their attachment characteristics and after washing with PBS cells detached from the plate. The adherence problems carried an increased variability of the experimental data affecting the final outcome, so experiments were repeated in different cell lines which kept their attachment properties.

Cell viability on HDFn cell line: HDFn cell lunes corresponds to new-born fibroblasts, cells were growth in DMEM, 10% FBS and present a doubling time around 30 h according to manufacturer. The first step to induce the growth factor induction protocol is to coordinate the cell cycle of the cells in the culture so the growth factor can induce a homogeneous effect in all the cells and get better results. To assess determinate the best coordination conditions, 2.500 cells were seeded con flat bottom 96-well plates in 100 μL, after 5 h of incubation the medium was replaced with DMEM supplemented with variable serum concentrations in order to evaluate cells stability in serum starving conditions. After 18 h of incubation with starving medium there are not significative differences with complete medium and they are capable of keep optimal conditions and stop cell cycle according to previous reports. These results can be related with the doubling time of the cell line because of what was expected to not see differences in cell number.

Once cell cycle coordination conditions were determined, cell proliferation in response to growth factors stimulation experiments were performed. Cells were stimulated with rhEGF and rhbFGF separated and together (24 h, 37° C., 5% CO₂). Cell number statistically increased in presence of 200 ng of each separated factor compared with non-stimulated cells inducing a quantifiable response. However, the signaling pathways involved are variable and the observed results correspond to only one of the possible responses to the growth factor stimulation. According to our model of action, the activation of this pathway could be enough to achieve a regenerative effect in salivary gland. To confirm these results, it is necessary to evaluate the effect of growth factors in other cell lines to have a better idea of the effects of the formulation in vivo.

Cell viability on MC3T3-E1 cell line: Cell line MC3T3-E1 corresponds to a pre-osteoblasts cell line usually used to cell differentiation assays but also phenotypic evaluation as cell proliferation can be made, so they were used for these experiments even if it's not the best option because these cells were available at the laboratory. These cells grow in alfa-MEM medium, 2 mM glutamine, 10% FBS without ascorbic acid to prevent the differentiation to osteoblasts. When the experiments previously performed on HDFn cells were made, rhEGF stimulation showed an increased cell viability for all conditions in about 50%, except when cells were treated with 50 ng/mL of rhEGF that the increment is of 86%, all the evaluated conditions show statistically significative differences compared to control. rhbFGF stimulation induces an increment according to data reported in literature of ≈150% of cell viability in presence of rhbFGF 5 ng/mL. All evaluated concentrations show a statistically significative increase compared to control (p<0.05). After stimulation with a mixture of both growth factors (5:1; rhEGF:rhbFGF) all the evaluated conditions show statistically significative differences compared to control.

Cell viability on MC-3T3-E1 cells by fluorescence microscopy: To complement the results obtained with PrestoBlue kit, living cells were observed by confocal fluorescence microscopy using a Live&Dead kit that allows to see live cells and dead cells. Flat bottom 24-well plates with glass covers on each well were seeded with 25.000 cells and the treatments previously described for cell viability were repeated, 24 h after the stimulation Live&Dead kit was prepared according to manufacturer instructions (45 min, 37° C., 5% CO₂).

Qualitative evaluation of the obtained images show that growth factors separated and together induce an increased cell number after 24 h, results that correlated to the data obtained when cell viability was assessed using PrestoBlue reagent.

Cell viability of loaded core-shell nanoparticles on MC-3T3-E1 cell line: After the effect of growth factors was determined, cells were stimulated with loaded core-shell nanoparticles (L EGF (AL-CH)₅) to determine the bioactive effect of the formulation, so far, only one assay was performed with different concentrations of nanoparticles following the methodology described above, as reference the total amount of growth factors in the formulation was used. The experiments show that all the concentrations evaluated increase cell viability, indicating the maintained bioactivity of the encapsulant within the core-shell nanoparticles.

Wnt-β catenin signaling pathway activation: In addition, experiments to determinate if the effect of the regenerative effect of the formulation is mediated by the convergence of FGF and EGF signaling pathways with Wnt pathway. According to the data presented by Hashimoto et al, 2002 the effects produced by FGF stimulation are, in part, mediated by β-catenin. The treatment activated the phosphorylation of GSK-3β which it's associated to a translocation of β-catenin to the nucleus, triggering the cellular response in different cell types, such as epithelial, fibroblasts, neurons and other cell types. In order to demonstrate that the observed effect is produced by the activation of a signaling pathway in response to the stimulation with growth factors and is not a secondary effect of the polymeric coating β-catenin localization was observed, since this should be translocated to the nucleus after the activation. To achieve this, cells were treated with recombinant growth factors (200 ng/mL, according to Wang et al. 2017), after 2 h of incubation two different experiments were performed: (1) western blot after lysis and separation of nuclear and cytoplasmic proteins and (2) Fluorescence microscopy of β-catenin.

Wnt-β Catenin signaling pathway activation by Western Blot: Cellular lysis, separation of nuclear and cytoplasmic proteins with NE-PER Nuclear and Cytoplasmatic Extraction kit. These preparations were used to assess the presence of the protein in each cellular compartment by western blot, using β-actin as loading control. β-catenin presence in nucleus and cytoplasm was measured for different treatments, qualitatively, is possible to observe a higher amount and intensity of the bands because of β-catenin degradation in nucleus this can lead to conclude that there is an increment in the amount of β-catenin translocated to the nucleus. However, even if there is a tendency to the accumulation of β-catenin, both in nucleus and cytoplasm, the differences observed are not statistically significative (P<0.05, one-way ANOVA). In the other hand, the observed phenotype (proliferation) is triggered as a response to the applied treatments, so it must be considered that this answer could correspond to an accumulative effect of the activation of different signaling pathways, and not exclusively to the activation of Wnt/β-catenin pathway. Given the accumulated knowledge and skill from this experience, the protocol is currently undergoing necessary adjustments to better determine the adequate amounts of antibodies, protein and stimulation time in order to then determine the amounts of our formulation that need to be added to the cells to trigger the activation of the pathway and, in consequence, to induce tissue regeneration.

Wnt-β catenin signaling pathway activation by immuno-fluorescence: β-catenin translocation was also assessed by immunofluorescence, β-catenin and β-actin antibodies were co-incubated in the samples after the treatment with the different formulations, it's possible to observe β-catenin localization, its translocation to the nucleus and the effect of treatments, white arrows indicate a slightly accumulation of fluorescence near to the nucleus which indicates the effect of the treatments and the communication between growth factors' signaling pathways and wnt/β-catenin pathway.

### EXAMPLE 5. IN VIVO BIOCHEMICAL AND HEMATOLOGICAL ANALYSIS

Surgical procedures: All related surgeries were performed at the experimental unit of CEMyQ Biotereo. First, animals were weighed for an initial analysis and then anesthetized by an experienced veterinary with an induction of vaporized isoflurane followed by the administration of ketamine (60 mg/kg of weight) and xylazine (8 mg/kg of weight). Once anesthetized a trichotomy in cervical region and asepsis and antisepsis protocols with alcohol 70% and povidone iodine were performed. A sub mento-sternal incision in the zone was made and a posterior divulsion in the anterior neck fascia. Salivary glands were localized with mosquito clamp to separate them from the proximal fascia and have free access to the puncture zone. After injecting the formulation, suspended in 100 μL (because of the glands size the volume can't be higher than that), the zone was sutured, and the animals were kept in normal conditions at bioterium facilities. The surgical follow up was made by the work team, animals had a regular diet. As part of this preliminary study, 1 μg of total growth factors in 25 μg of nanoparticles (L EGF (AL-CH)₅ FGF, (NP-GFs)) suspended in a final volume of 100 μL of PBS. The controls of the experiment were unloaded nanoparticles (L (AL-CH)₅, 25 μg (NPs)), each growth factor separated (1 μg/100 μL), uncoated liposomes and PBS.

Preliminary results: Each animal was analyzed weekly and saliva samples were collected. To obtain saliva samples, animals were sedated and positioned vertically in stable process in 15-25 minutes time lapses, saliva was collected in Eppendorf tubes; this process was performed every week and the samples freeze until analysis. During the first week there are not major observable variations in any of the evaluated groups. However, in second and third week, it is possible to see a tendency in the group injected with the formulation to produce a greater amount of saliva compared to the other groups, control animals produced 0.02 mL of saliva and treated animals 0.28 mL at third week, reaching a 1400% of saliva production in the group treated with NPs+EGF+bFGF compared to those injected only with vehicle (PBS/control). Salivary collection for quantitative analysis, primarily, and qualitative analysis, later on, was a challenge. Nonetheless, we've even tried to collect un-stimulated (as in original proposal and study design) as well as stimulated (following discussions with committee at Conicyt) mainly due to the custom-made irradiation device and exposure deemed significantly severe and damaging (dry mouth) to the size as well as species of the mice. Here in, despite the above challenges, we have successfully collected enough quantity of saliva to compute a significant 1400% increase in salivary production 3 weeks post-IR in animals injected with the composition of the invention, when compared to control animals injected with PBS. All animals were injected, directly and bi-laterally into the surgically-exposed sub-mandibular glands, 1 day before IR. Saliva was collected intra-orally and also from the ducts themselves. Furthermore, we have successfully managed to study the biochemistry of the collected saliva, reporting on values of cholesterol, glucose, and enzymatic content.

On the other hand, at the moment of euthanasia, blood samples were obtained to measure the most common parameters to assess the condition of the major organs as liver, kidney, etc. The analysis of this stage was performed along with plasma studies, the obtained results are shown in the next section.

Irradiation model: The original plan in this project, was to utilize a clinical center for humans, authorized with radiotherapy normal use equipment, this was not possible because of changes in the regulation for certification of hospital centers. Because of this, a joint work was carried out with Medical Physics department in order to fulfill the requirements of this investigation. The project team were faced by the challenge/obstacle of finding a location that allowed the irradiation of animals, without success. Hence, they were faced with the next challenge of securing an irradiation system, which also failed. Finally, the team (assisted by others outside the project) opted to assemble a custom-made (in-House) irradiation system composed of a portable X-ray generator (CP160D, Teledyne ICM) equipped with a dynamic operation range from 10-160 kV, and a maximum current of 9 mA, maximum power of 900 W and continuous irradiation windows without time limit (plus a filter carousel for variable irradiation fields size). The system is remote controlled by an external PowerBox digital console that allows to tune in increments of 1 kV in potential difference, 0.1 mA of tube current and increments of irradiation temporary time windows of 1 s. Supplied dose and irradiation time were determined through a Farmer ionization chamber (A19, EXRADIN), the equipment has valid calibration for 30-200 kV orthovoltage energy beams. Dosimetric setting was done establishing surface input (because of low depth of the irradiated organ and the absence of certificate phantoms for the needed application) and considering a retroduction factor of 1.4 to the total absorbed dose, according to the values obtained from Arcal dosimetry protocols for imageology equipment, considering the quality of the beam obtained with the equipment working in 160 kV and an intrinsic 3 mm Al filter. The chamber was placed with its buildup cover at real irradiation distance (70 cm) with the equipment operating in a field wide enough to completely cover the ionization face. The current was set in 5 mA during all the measurement period. With this configuration a dose entrance rate in the central axis of 105 mGy/min was obtained. Side dose measurements were made in order to determine dose variation outside the axis with variation lower than 10% for a distance of 20 cm outside the axis. Considering backscatter factors, it was established that the necessary time to achieve an absorbed dose of 4.5 Gy was 30 min 45 s. Irradiation system is contained in a specially designed housing that accomplish Safety Reports Series No. 58—IAEA Publications y Safety Reports Series No. 47—IAEA Publications regulations that rule right radiological protection and exposure of irradiation equipment operators. Irradiation system internal dimensions are 1.0×1.0×1.8 m³, allowing to irradiate different biological samples, inorganic samples and small and medium size animals, according to needs. The system is placed in an optic table designed for irradiation that ensures stability , of the samples and holds the almost 1000 kg required blindage. In order to limit the supplied dose exclusively to target region a full body collimator was built for the mice set that will be irradiated simultaneously. Collimator has 8 mm of lead thickness allowing a 2 cm field on mice interest region. Irradiation system was aligned systematically in each irradiation by laser alignment guides for mice to be placed symmetrically to central axis to get equivalent doses. Immediately after irradiation process and after anesthetic effect passed animals were unappetizing and weak until 24 h after irradiation, when they resume normal food and water intake. Damage and irradiation confirmation were evidenced from day 15 post-irradiation. Experimental animals suffered hair loss in irradiated zone and around the area. On the other hand, salivation decreased to levels rendering it very difficult to collect saliva samples despite the many collection methodologies used.

Biochemical serum analysis: To perform serum analysis, serum was separated through centrifugation (3500 rpm, 15 min.) and stored at −80° C. until use. The following analytes were evaluated: Chlorine, Magnesium, albumin, creatinine, total protein, total cholesterol and triglycerides (colorimetric method with automatic spectrophotometer, HUMAN Diagnostics Worldwide, Wiesbaden, Germany). Enzymatic activity of alkaline phosphatase (ALK), alanine transaminase (ASAT) were all analyzed through a colorimetric enzymatic assay (automatic spectrophotometer, HUMAN Diagnostics Worldwide, Wiesbaden, Germany).

TABLE 5

| | Media DS | |
|---|---|---|
| | Cloro | Fosforo |
| | (mg/dL) | (mg/dL) |
| unIR + NPS | 392.34 ± 2.59 | 7.70 ± 0.83 |
| unIR + EGF | 394.05 ± 1.05 | 8.45 ± 0.36 |
| unIR + EGF + NPS | 400.13 ± 3.87 | 11.61 ± 6.60 |
| unIR + bFGF | 398.88 ± 1.15 | 7.67 ± 0.25 |
| unIR + bFGF + NPS | 405.10 ± 20.26 | 7.23 ± 1.12 |
| unIR + EGF/bFGFpure | 395.73 ± 2.19 | 7.22 ± 1.16 |
| unIR + EGF/bFGF + NPS | 399.10 ± 0.99 | 8.60 ± 0.20 |
| IR + NPS | 398.86 ± 3.32 | 9.23 ± 1.04 |
| IR + EGF | 398.26 ± 3.29 | 7.48 ± 0.53 |
| IR + EGF + NPS | 396.88 ± 1.01 | 7.92 ± 0.78 |
| IR + bFGF | 398.85 ± 1.45 | 7.95 ± 0.49 |
| IR + bFGF + NPS | 397.20 ± 3.05 | 18.08 ± 19.76 |
| IR + EGF/bFGFpure | 394.23 ± 5.09 | 8.37 ± 0.72 |
| IR + EGF/bFGF + NPS | 394.36 ± 3.12 | 7.19 ± 0.60 |

Plasmatic electrolytes of C57BL/6 mice exposed to growth factors against damage and deterioration of salivary glands
after gamma irradiation in head and neck region
^{¶}At least one group is different than other (p < 0.05),
ª Statistically significative differences with unIR + NPs group,
^{b} Statistically significative differences with unIR + EGF group,
^{c} Statistically significative differences with unIR + EGF + NPs group,
^{d} Statistically significative differences with unIR + bFGF group.

TABLE 6

| | Media DS | | | | | |
|---|---|---|---|---|---|---|
| | | Fosfatase | | | | Total |
| | Albumin | Alcaline | Creatinin | GOT | GPT | protein |
| | (g/dL) | (U/I) | (mg/dl) | (g/dL) | (g/dL) | (g/dl) |
| unIR + NPS | 2.79 ± 0.21 | 180.02 ± 18.61 | 1.94 ± 0.022 | 67.40 ± 21.11 | 32.60 ± 6.99 | 3.86 ± 0.23 |
| unIR + EGF | 2.92 ± 0.13 | 169.30 ± 20.92 | 0.14 ± 0.02 | 107.00 ± 55.53 | 34.40 ± 9.76 | 4.10 ± 0.19 |
| unIR + EGF + NPS | 3.02 ± 0.25 | 195.03 ± 31.42 | 0.54 ± 0.58 | 79.33 ± 34.79 | 27.33 ± 7.57 | 4.10 ± 0.22 |
| unIR + bFGF | 3.05 ± 0.22 | 180.77 ± 20.95 | 0.23 ± 0.02 | 90.61 ± 36.73 | 32.00 ± 13.55 | 4.12 ± 0.28 |
| unIR + bFGF + NPS | 2.82 ± 0.27 | 156.02 ± 19.69 | 0.19 ± 0.03 | 88.33 ± 58.70 | 26.00 ± 7.65 | 4.03 ± 0.49 |
| unIR + EGF/bFGFpure | 2.66 ± 0.20 | 208.62 ± 36.52 | 0.17 ± 0.01 | 87.00 ± 19.94 | 46.20 ± 20.27 | 3.95 ± 0.32 |
| unIR + EGF/bFGF + NPS | 3.02 ± 0.12 | 161.75 ± 33.45 | 0.30 ± 0.02 | 92.33 ± 45.00 | 19.50 ± 6.36 | 4.23 ± 0.15 |
| IR + NPS | 2.82 ± 0.09 | 176.18 ± 21.51 | 0.17 ± 0.04 | 92.20 ± 44.06 | 32.80 ± 7.60 | 3.86 ± 0.05 |
| IR + EGF | 2.92 ± 0.24 | 181.27 ± 22.13 | 0.19 ± 0.03 | 87.50 ± 21.68 | 31.17 ± 4.71 | 3.98 ± 0.24 |
| IR + EGF + NPS | 2.74 ± 0.12 | 183.80 ± 10.35 | 0.19 ± 0.02 | 77.80 ± 27.73 | 25.80 ± 4.15 | 3.80 ± 0.19 |
| IR + bFGF | 2.84 ± 0.11 | 180.93 ± 34.54 | 0.16 ± 0.02 | 68.75 ± 13.3 | 26.75 ± 10.44 | 4.03 ± 0.13 |
| IR + bFGF + NPS | 2.26 ± 0.74 | 141.45 ± 62.69 | 1.57 ± 2.71 | 62.75 ± 55.87 | 53.75 ± 47.95 | 3.35 ± 0.90 |
| IR + EGF/bFGFpure | 2.51 ± 0.06 | 157.27 ± 16.45 | 0.22 ± 0.05 | 64.00 ± 9.54 | 27.00 ± 2.65 | 3.57 ± 0.21 |
| IR + EGF/bFGF + NPS | 2.71 ± 0.06 | 159.60 ± 7.49 | 0.14 ± 0.01 | 54.20 ± 15.09 | 22.00 ± 4.00 | 3.76 ± 0.05 |

Biochemical profile of C57BL/6 mice exposed to growth factors against damage and deterioration of salivary glands after gamma irradiation in head and neck region

TABLE 7

| | Media DS | |
|---|---|---|
| | Colesterol Total | Trigliceridos |
| | (mg/dL) | (mg/dL) |
| unIR + NPS | 63.80 ± 8.14 | 37.25 ± 14.15 |
| unIR + EGF | 67.00 ± 9.97 | 26.33 ± 4.16 |
| unIR + EGF + NPS | 73.33 ± 8.08 | 43.67 ± 6.43 |
| unIR + bFGF | 66.80 ± 6.22 | 52.00 ± 0.00 |
| unIR + bFGF + NPS | 70.40 ± 12.24 | 48.75 ± 21.56 |
| unIR + EGF/bFGFpure | 63.20 ± 4.21 | 51.50 ± 23.81 |
| unIR + EGF/bFGF + NPS | 68.00 ± 4.24 | 35.00 ± 1.41 |
| IR + NPS | 66.80 ± 8.53 | 65.00 ± 8.57 |
| IR + EGF | 64.00 ± 14.83 | 54.40 ± 27.37 |
| IR + EGF + NPS | 67.60 ± 18.62 | 56.20 ± 23.58 |
| IR + bFGF | 59.25 ± 8.22 | 42.25 ± 6.40 |
| IR + bFGF + NPS | 57.00 ± 18.96 | 55.75 ± 16.76 |
| IR + EGF/bFGFpure | 67.67 ± 5.51 | 59.00 ± 6.24 |
| IR + EGF/bFGF + NPS | 64.40 ± 4.16 | 48.20 ± 12.91 |

Lipidic profile of C57BL/6 mice exposed to growth factors against damage and deterioration of salivary glands after gamma irradiation in head and neck region

### EXAMPLE 6. EVALUATION OF RADIO-PROTECTOR EFFECT: HISTOMORPHOMETRICS

Quantitative RT-qPCR assays and Apoptotic response of irradiated submandibular glands from paraffin-embedded tissue sections are evaluated via in situ terminal deoxynucleotidyl transferase-mediated dUTP nick-end labeling (TUNEL).

### Quantitative RT-qPCR Assays:

RNA extraction: RNA was extracted from salivary glands stored at −80° C. with the reagent TRIzol® (Life technologies). The samples were placed in a tube with 1 mL of cold TRIzol and tissue was homogenized with a pestle homogenizer, manufacturer instructions were followed.
RNA quantification and integrity evaluation: Extracted RNA was quantified by fluorimetry with the kit Qubit RNA BR assay kit (Invitrogen) and integrity (RIN) with Qubit RNA IQ assay kit, both evaluations were measured in QUBIT4.0 equipment (Life technologies).
Reverse transcription and qPCR: RNA was converted to cDNA from 1 μg ARN through reverse transcriptase according to manufacturer recommendations with High capacity RNA to cDNA Kit (Applied biosystems). Then real time PCR was performed for the quantification of relative expression of axin 2 (Axing), aquaporin 5 (Aqp5) and vascular endothelial growth factor a (VEGFa) in the equipment QuantStudio 3 (Applied biosystem) and the expression evaluated with the Livak 2-^{ΔΔCt} method with the normalizer gene GADPH.
Results: To perform this analysis, groups of interest were studied to establish the concentration and integrity evaluation of RNA (RIN) in experimental groups (irradiated vs. not irradiated; with growth factors EGF & bFGF and without (blank NPs); and with/without nanoparticles it's shown in Table From all the extracted samples was obtained high quality, integral RNA, to perform real time PCR studies.

TABLE 8

| | | Concentration ARN | |
|---|---|---|---|
| Groups | Biological replica | μg/μl | RIN |
| IR EGF-bFGF Pure | Mouse 1 | 1.96 | 7.8 |
| | Mouse 2 | 1.09 | 6.7 |
| | Mouse 3 | 2.12 | 7.7 |
| IR EGF-bFGF NPS | Mouse 1 | 1.32 | 8.5 |
| | Mouse 2 | 1.17 | 8.4 |
| | Mouse 3 | 1.66 | 8.5 |
| UIR EGF-bFGF Pure | Mouse 1 | 2.04 | 8.0 |
| | Mouse 2 | 2.28 | 7.7 |
| | Mouse 3 | 2.04 | 8.2 |
| UIR EGF-bFGF NPS | Mouse 1 | 2.36 | 7.3 |
| | Mouse 2 | 2.28 | 8.9 |
| | Mouse 3 | 1.99 | 8.2 |
| IR SALINE | Mouse 1 | 1.02 | 8.6 |
| | Mouse 2 | 1.98 | 8.0 |
| | Mouse 3 | 1.2 | 8.2 |
| UIR SALINE | Mouse 1 | 2.16 | 7.5 |
| | Mouse 2 | 2.12 | 7.5 |
| | Mouse 3 | 2.24 | 7.6 |

Concentration and RIN of RNA extracted from salivary glands of irradiated and no irradiated mice. IR EGF-bFGF NPS: Irradiated mice injected with Epidermal Growth Factor and Fibroblast Growth Factor b encapsulated in nanoparticles, UIR EGF-bFGF NPS: : No irradiated mice injected with Epidermal Growth Factor and Fibroblast Growth Factor b encapsulated in nanoparticles, IR EGF-bFGF Pure: : Irradiated mice injected with Epidermal Growth Factor and Fibroblast Growth Factor b, UIR EGF-bFGF Pure: No irradiated mice injected with Epidermal Growth Factor and Fibroblast Growth Factor b, IR Saline: Irradiated mice injected with saline solution, UIR Saline: No irradiated mice injected with saline solution.

No statistically significant differences were found in the expression of axin 2 (Axin2), aquaporin 5 (Aqp5) and Endothelial Vascular Growth Factor a (VEGFa) genes among the studied experimental groups.

It is important to highlight that the in the project and platform was compromised to inform the values of MDA, a widely use marker of lipid peroxidation. Since the extra funds destined to the implementation of the irradiation system the number of measured markers was reduced and MDA was not measured. Due to the problems that have been recently reported for the colorimetric assessment of MDA is that arose the need of buy expensive kits or technics (like HPLC) to obtain accurate values of the marker, and the questions about its specificity as oxidative stress marker (Khoubnasabjafari, 2015 and Tsikas, 2017) lead to decide to eliminate this marker and gave priority to the marker Axin2 (that was reported in the platform as a replacement of MDA). Axin2 raises as a more useful parameter because more information can be inferred, directly and indirectly, from its values. Axin2 is upregulated when wnt-β-catenin pathway its activated, also is a negative regulator of this pathway (Jho, 2002) and has been describe as a marker of wnt-β-catenin activation (Yan, 2001), pathway that have been suggested to be part of the radioprotection mechanism (Wang, 2013). So, the levels of Anxin2 will provide an evidence of the radioprotective effect of our formulation not by cell damage, as MDA would do as an oxidative stress marker, but representing the activation of wnt-β-catenin pathway. Besided, β-catenin is regulated by ROS (Funato, 2006), so it's also an indirect indicative of the oxidative status of the tissue. Finally, Axin2 have also been linked to cancerogenic processes (Wu, 2012). In conclusion, the assessment of MDA was replaced by a marker that can be measured easily and economically by qPCR and that can provide more information about the effects of the formulation in salivary glands. Axin2 didn't show significative differences, not indicating and activation or inactivation of wnt-β-catenin pathway which could imply that there are not oxidative or cancerogenic processes induced by the formulation.

Conclusions: Aqp5 is frequently found present in salivary glands and the absence of differences here in indicates that cellular characteristics did not have any modifications with the applied experimental product. No differences in Axin2 could be expected since the animals didn't present oncological changes in their system. VEGFa is a factor involved in angiogenesis, vasculogenesis and endothelial growth; in our samples were not detected differences in this analysis, probably because that the application of the product lead to an increased in the size of the excretory portion of the gland, not related to regional vascularization increments.

### EXAMPLE 7. EVALUATION OF RADIO-PROTECTOR EFFECT: HISTOPATHOLOGICAL ANALYSIS OF MAJOR ORGANS

The brain, liver, lungs, kidneys, heart and spleen will be removed from each animal at time of euthanasia, stained with H&E and processed for comparative histo-pathological examination via light microscopy and rated by blinded expert evaluators for any signs of inflammation and/or infection.

Macroscopic analysis of organs: Animals were euthanized with an overdose of ketamine and xylazine, after mentioned time. Organs were collected from each animal, including salivary glands, lungs, kidney, liver and brain. The determination of organs weight was made through Scherle method. Data obtained from the evaluation of other organs according to its weight is useful as an indicator of potential alterations (safety/biocompatibility validation of final product, its components and by-products) that an organ can suffer in response to the stimulus mediated by the injection. All the organs presented normal macroscopic characteristics without mass or appearance alterations. To complete this analysis, a histological analysis of the samples was performed, results are shown below.

Histological and stereological analysis: Mice have 3 principal salivary glands paired that are visible macroscopically: sublingual, parotid and sublingual; and numerous microscopic minor glands

Processing and staining: Salivary glands were weighed with analytic balance (Radwag WTB 2000) and fixed with 4% buffered formalin (1.27 mol/L formaldehyde in phosphate buffer 0.1 M pH 7.2) for 48 h, dehydrated and embedded in Paraplast Plus. Once blocks were obtained the were cut with 5 pm thickness (Leica® RM2255) and stained with HE and alcian blue stainings for its histological and stereological analysis. To perform this analysis salivary gland (HE, PAS, alcian blue and picrosirius staining), liver (HE), kidney (HE) and brain (Nissl staining) were analyzed. Reagents: Pricosirius (Sigma Aldrich, Sant Louis, Missuri, USA), PAS (Darmstadt, Germany) and HE (Darmstadt, Germany). The utility of each staining is described below:
(a) PAS (periodic acid-Shift): Technique that stains purple neutral mucosustances; stains goblet cells. Used to, for example, evidence metaplasia.
(b) Picrosirius: Yellow-Red color associated to type I collagen and green color is associated to type III collagen; very useful in salivary gland analysis, especially in the study of stroma.
(c) HE: Hematoxylin (cationic), stains acidic structures (basophil) in blue and purple, for example, cell nucleus; eosin stains the basic components (acidophilus) in pink, thanks to its anionic or acid nature, cytoplasm. Commonly used in cellular description.

Stereological analysis: All stereological analyses were performed in 5 animals of each group. Duct analysis was made via examining six fields in each animal with 30 total fields per group (Mandarim-de-Lacerda & del Sol, 2017). To develop stereological analysis of acinar cells 22 fields per animals were examined and 110 total fields in each group. Slides were observed in stereological microscope (Leica® DM2000 LED) and photos took with digital camera (Leica® MC170 HD) and analyzed using multipurpose M42 system. In ducts the following parameters were determinate: number density/area (N_{Acond}), volume density (V_{Vcond}) and surface density (S_{Vcond}). For acinar cells the following parameters were evaluated: number density (N_{Vacin}) and volume density (V_{Vacin}). Number density/area of ducts (N_{Acond}) was determinate according to the equation: N_{Acond}=N/A_{T} (N: number of observations in a delimited area considering prohibited lineas and A_{T}: total area of the system (36.36×d²) d²: lines of the test system with known longitude). Ducts volume density (V_{Vcond}) was assessed applying the following formula: V_{Vcond}/P_{T} (100%) (P_{Pcond}:number of points were conducts touch and P_{T}: total number of system points). Ducts surface density (S_{Vcond}) was determinate according to the following formula: S_{Vcond}=(2×I)L_{T} (I: number of intersections that touch the structure and L_{T}: total length of M42 system test lines). Also, acinar cells number density (N_{Vacin}) was evaluated according to the equation: N_{VAcin}=Q⁻/(A_{T}×t) (Q⁻: number of observations in a determinate area considering forbidden lines and plane and t: cut thickness).

Histological characteristics of submandibular salivary glands in all groups present an architecture within normal limits and did not show pathological/abnormal differences among groups. The following figures are constructed with representative images of each group. The mouse presented a pair of submandibular glands that structurally corresponded to composed acinar type. Its parenchyma was divided through partitions of connective tissue, in lobs and lobes that allowed the entrance of vessels and nerves. Acines were of mixed type, with serous predominance. Mixed acines were scarse, they appear as mucous cells partially surrounded by semi moons of serous cells. Serous cells presented a pyramidal aspect, with a wide basal surface and round nucleus. In the basal zone of cytoplasm, it's possible to observe zymogen granules stained with hematoxylin after HE conventional staining. Mucous cells are more rounded and its nucleus, of basal location, are more flattened. Cytoplasm with mucinogen granules was slightly stained with HE is staining but they are more evident with carbohydrate staining as PAS and alcian blue. At intralobular level it's possible to observe intercanal ducts, granulated, contoured and fluted, homogeneously distributed in the glandular parenchyma. They were constructed by epithelial cells with morphology that gradually varies from cubical to cylindrical. Granular contoured and fluted ducts were prominent, they were coated with cubic cells with abundant eosinophilic cytoplasm. Granular ducts were characterized by presenting eosinophilic granules in its cytoplasm. The most evident excretory ducts were the fluted intralobular ducts. They are bigger and surrounded by abundant collagenous connective tissue. These ducts were coated with cubic or cylindric cells with round nucleus of more central localization.

Un-Irradiated group (un-IR): Related to the submandibular salivary gland, histologically presented an architecture within normal limits. Glandular tissue was organized in a multi- and lobulated way. Parenchyma was tubulo-alveolar mixed with serous predominance that was connected through ducts. In male mice, glandular secretion is excreted first through intercalar ducts of very small lumen, then through granular contoured ducts, intralobular ducts and finally through interlobular ducts. The latest merge in excretory ducts that flow into the oral cavity. It's important to highlight that the granular contoured ducts were coated with big and columnar cells that contained prominent eosinophilic granules and the intralobular ducts by simple cubic epithelium whose cells presented nucleus with central location. In glandular tissue, serous cells presented a pyramidal appearance, with a wide basal surface and round nucleus. In the basal zone cytoplasm shows zymogen granules stained with hematoxylin. Mucous cells were more rounded and their nucleus, of basal location, more flattened. The cytoplasm with mucinogen granules ere slightly stained with H&E staining. The staining with periodic acid-Schiff (PAS) and Alcian Blue pH 2.5 (AA pH 2.5) showed the presence of neutral and acidic mucopolysaccharides, respectively, in glandular tissue and secretion products. But nevertheless, the cells in excretory ducts didn't show positive reaction.

Experimental un-irradiated group: When compared to experimental groups with un-IR control group, generally, differences in histological structure of the gland were not observed, so, these groups also presented a conserved structure within the normal limits, both at the level of parenchyma (adenomeres and ducts) stroma-like. At the moment of comparing the experimental groups, to which growth factors were administered, with its corresponding NPs groups, neither showed differences in the histological characteristics. After applying the protocol, some changes were observed in the granular contoured ducts, those who presented a slight hypertrophy in the un-irradiated groups with growth factors, compared to the control group un-IR-saline. The stereological analysis showed a higher length density (Lv) in the groups with growth factors than this group, even though without significative values. The surface density (Sv) and volume (Vv) didn't show significative differences among un-irradiated groups. Staining with PAS and AA pH 2.5 showed a higher secretion of the glandular adenomeres in the groups with growth factors. But, nevertheless no a higher number of acinar cells (Nv) or an increase in their percentage was observed (Vv).

Irradiated experimental groups (IR): When irradiated groups were compared, to which it was administered growth factors, with and without NPs, with internal controls of the experimental phase (IR-saline and IR-NPs) it was possible to observe some differences. Adenomeres and granular contoured ducts showed hypertrophy and slight hyperplasia compared to controls IR-saline and IR-NPs. Staining with PAS and AA pH 2,5 showed a higher secretion from glandular adenomeres in the groups with growth factors, but mild to moderate.

As noted above, was corroborated through the stereological analysis, although without significative differences. All the irradiated groups to which the growth factors were administered, with and without NPs, showed a higher number of acinar cells per cubic millimeter (Nv) than their controls (IR-saline and IR-NPs). Likewise, IR-bFGF+NPs, IR-EGF+bFGF+NPs showed an increase in the values of Lv and Sv in the ducts compared to controls, which is evidenced in higher length and size per cubic millimeter, respectively.

Unirradiated vs. Irradiated: In a general context, when gland from the un irradiated groups are compared with irradiated, no histological and stereological differences were observed in the secretory portion. There were no histological differences in the stromal connective tissue. Nevertheless, the excretory portion showed significative differences. Irradiated groups bFGF+NPs and EGF+bFGF+NPs, showed values significative higher for Lv in ducts than un irradiated groups and the groups bFGF+NPs, EGf+bFGF and EGF+bFGF+NPs showed higher values of Sv in ducts than un irradiated groups. This reflects an increase in length and surface per cubic millimeter of the ducts in this groups.

Conclusions and remarks: According to the obtained results in this in vivo study, there are several important considerations that can be highlighted. (i) None of the injected formulations had any negative consequences on animal's health, they didn't affect behavior, weight or food intake. Therefore, harmlessness/biocompatibility of the formulation is demonstrated in the animal model. (ii) Histological analysis show that tissues remain in a normal state, both in salivary glands as in other major organs such as brain, liver and kidney. (iii) Watching at the amount of secreted saliva, is possible to observe that 2 or 3 weeks after injection, there is a tendency of the group injected with the composition of the invention LEGF(AL-CH)₅FGF, which agrees with the proposal and scientific formulation of the project, nanoparticles allow a controlled release of growth factors that can induce a positive protective and reparative effect in glandular tissues.

So, it can be concluded that signaling pathway(s) is/are activated thereby stimulating tissue regeneration and, in consequence, a higher production of saliva.

### Histological and Stereological Analysis:

Unirradiated groups: Submandibular glands of un-irradiated mice to which growth factors were administered didn't present histological or stereological differences in the secretory and excretory portions compared to control groups. There were no histological differences in stromal connective tissue. Histological analysis showed that the glandular ducts of submandibular glands in un-irradiated mice, to which growth factors were administered presented mild hypertrophy. These changes could be related with a, no significative, increase in the length per cubic millimeter (LV) of glandular ducts. NPs didn't change or affect the normal architecture of submandibular gland of male un-irradiated C57BL/6 mouse. Growth factors produced a slight increase in PAS and AA pH 2.5 staining in glandular adenomeres in un-irradiated groups.

Irradiated groups: Submandibular glands of irradiated mice to which growth factors were administered presented some histological differences with their respective controls. They showed mild hypertrophy and hyperplasia, both in adenomeres and in glandular ducts. There were no histological differences in stromal connective tissue. Stereological analysis showed that there were no statistically significative differences among the different groups of irradiated mice. Thus, growth factors don't change or affect the stereological characteristics of acinar cells and glandular ducts from the submandibular gland of irradiated male C57BL/6 mouse. NPs did not change or affect the normal architecture of male C57BL/6 submandibular gland of irradiated male mouse.

Unirradiated vs. Irradiated: In a general context, when glands from un-irradiated and irradiated groups are compared, no histological or stereological differences in the secretory portion were observed. There were no histological differences in stromal connective tissue. The growth factor bFGF incorporated in NPs and the mix of EGF and bFGF pure or in NPs, act on the glandular ducts stimulating a higher length and surface of ducts when submandibular glands are irradiated. NPs didn't change or affect the normal architecture of male C57BL/6 submandibular gland of irradiated and un-irradiated male mouse. In vivo analysis of the core-shell formulation injected directly into salivary glands before head and neck region irradiation showed an important effect in salivary gland radioprotection verified by biochemical, stereological and histological analysis. The obtained values are similar and better to those obtained with pure growth factors which indicated an early effect of our formulation, even when growth factors' released amounts are just a small percentage of total loaded factors. Hence, this protective and potentially-reparative and regenerative effect can be expected to be maintained and improved on time with core-shell delivery system since the time-sustained controlled-released of growth factors that characterizes our drug delivery system. Indeed, the presented in vitro release kinetics profiles of the core-shell nanoparticles demonstrated a sequential/ordered, continuous/sustained and linear release period up to 55 days (at least, without reaching a plateau), with accumulative concentrations of available growth factors, results correlated with previously publish data by the principal investigator in 2008. The controlled release kinetics of our formulation will allow to achieve higher concentrations of active growth factors in extended periods of time, opposite to pure growth factors, since they don't have a delivery system that protects their bioactivity and bioavailability or modulates their release kinetics and so, the factors, even if remain active, will be available for much shorter periods of time (known to have a short half-life). It is because of this that our formulation is expected that, besides the observed early radioprotective effect, will have an improved late effect in comparison to the direct injection of growth factors, in extended periods of time, due to the release of factors from our system is slow and controlled, and can be fine-tuned/modulated as per the requirements of the clinical application/indication.

Histopathological analysis of other organs: Using the methodology previously described for the analysis of salivary glands, and hematoxylin-eosin staining to describe and compare tissues, selected organs were analyzed for different groups of interest. Evaluated organs were kidney, liver, spleen and brain cortex. Generally, the descriptive analysis confirmed the absence of alterations in the analyzed organs; in the same way, organs of animals included in experimental groups were selected to perform statistical comparisons, getting an absence of relations between the different groups. None of the comparative analysis between organs showed significative differences among organs of experimental groups.

### EXAMPLE 8. TUMOR AGGRESSIVENESS ASSESSMENT OF DUAL-RELEASE DRUG DELIVERY SYSTEM

Evaluation of tumor aggressiveness after the injection of formulation, the tumorogenic potential of core-shell nanoparticles containing growth factors will be determinate in vitro through clonogenic and tumorosphere assays and in vivo by the establishment of an orthotopic tongue cancer model in immunocompromised mice. This work is performed at BioMAT'X labs, UANDES (and Cells for Cells Company).

Tumor aggressiveness determination by clonogenic assays: To develop this assay, 500 cells were seeded in 35 mm plates in DMEM/F12 medium, 10% FBS, hydrocortisone and incubated 24 h (37° C., 5% CO₂). Plates were irradiated with 8 Gy intensity in Comision Chilena de Energia Nuclear facilities. Since pre-irradiation and post-irradiation treatments have been studied for radioprotection (Contrim, 2007; Houchen, 1999) we decided to test the effect of our formulation before and after irradiation, so cells were treated according of two experimental approaches: (i) Treatment with formulation before irradiation and (ii) Treatment after irradiation. 4 experimental conditions were tested (a) Nanoparticles loaded with growth factors (NP+GFs), (b) Unloaded nanoparticles (NP), (c) rhEFG+rhbFGF 50:50, 100 ng/mL of total proteins (GFs) and (d) Normal growth medium (CTRL+).

Data analyzed using imageJ, where the macro “ColonyArea” was used. In this way it was possible to obtain the Area percent (percentage of the well area that is covered with cells) (Guzman, 2014). and Intensity percent (cell density proportional staining intensity) of the clonogenic assay. The assay was performed in 3 different cell types: SCC-9, HDFn and MC3T3-E1. Clonogenic assay shows no statistically significative colony formation respect to control when treated with different conditions in most treatments, except when MC3T3-El cells were treated with our formulation that the colony formation showed to be significative minor compared to control. This data supports that loaded or unloaded nanoparticles doesn't increase tumor aggressiveness in vivo on the 3 cell types tested.

Tumor aggressiveness determination by tumorospheres assays: Since this experiment correspond to an extra-request from committee it involved the implementation and standardization of the method in our laboratory, because of that the first approach was to determinate the optimal number of cells and incubation time to obtained optimal data, to do this SCC-9 cells were starved overnight (no serum in F12 cell culture media). The next day, 200 or 500 individual cells (filtered through a 40 um cell strainer) were seeded in each well of a 96 wells low-binding cell culture plate. Treatments were performed as described, and formation of spheres was evaluated after 7- and 14-days post treatment. Quantification was performed by taking 4 photographs of each well with a 4× microscope objective, and images were quantified using imageJ, marking as “spheres” groups of more than 3 cells (300 pixels area). Tumoral media: 20 ng/mL EGF, 10 ng/mL FGF, 5 ug/mL insulin, 4 mg/mL BSA in F12 cell culture medium. The experiment shows that tumoral media is able to induce the formation of spheres, contrasting the observations at 7- and 14-days post treatment. However, in presence of excess Growth Factors (GFs), or GFs-loaded nanoparticles, the tumor spheres formation is not induced. After changing the number of cells to 500 individual cells, it's possible to observe that tumoral media is able to induce the formation of spheres, rendering it suitable for this assay. However, in presence of excess Growth Factors (GFs), or GFs-loaded nanoparticles, the tumor spheres formation is not induced.

The data above corresponds to the standardization process results, after adjusting cell number to 500 and incubation time to 14 days, the experiments were repeated with 3 cell lines, SCC9, HDFn and MC3T3-E1. In this set of experiments where used nanoparticle batches with slight improvements on the protocol that caused that the final treatments solutions had higher concentrations of nanocapsules, and with this of polymers in order to get the necessary growth factors concentration, which accumulated in the surface of the well obstructing the vision of the bottom where tumorospheres should be formed not allowing to quantify. As the plates needed for this kind of assays are low-binding remove the supernatant could pull the spheres with it. Different strategies for quantification were assayed, like fluorescent dyes (which stained all the materials on the well, so tumorospheres were not identifiable for counting; data no showed). New methods will be assessed in the future; however, tumor aggressiveness was assessed in two other different experiments, in vitro and in vivo so a part of the information that this assay will provide is covered by those.

Tumor aggressiveness determination in vivo: To determine tumor aggressiveness in vivo, an orthotopic tongue cancer model was induced in NSG mice, through the injection of SCC-9 cells. A preliminary pilot is progress to set the time and number of cells needed to produce a tumor in the animals. Animals were weighed, anesthetized with inhalator isoflurane and divided in two experimental groups (i) injection in the flank and (ii) injection in the tongue, 2 animals for each group. Animals of the group that was injected subcutaneously in the flank, received 2.5×10⁶ SCC-9 cells contained in 300 μL of PBS, one mouse was injected with the total volume in the right flank and the second one was injected in both flanks with 150 μL each (1.15×10⁶ cells in every injection). Mice corresponding to the group that was injected in the tongue, one was injected with 2.5×10⁶ SCC-9 cells in 40 μL of PBS and the other with 40 μL of PBS as control. Animals will be followed up until tumors formation. The animal corresponding to tongue tumors induction, presented the injection area swollen for days in the injection site, leading to a quick weigh lost due to the impossibility of food and water intake. The animal was supported with fluids and corticoids in order to reduce inflammation and weight loose. However, despite these efforts, the animal presented an important weight reduction and behavioral signs of stress, so it was euthanized 3 days after the injection. Because of the important damage for animals that implied the tongue model and considering the positive results obtained from the pilot in the animals injected in the flank is that the model was changed to a model of tumors in the right flank. 20 NSG mice, male and female, 7-9 weeks were inoculated with 1×10⁶ SCC-9 cells in 100 mL of sterile PBS 1× at the right flank. The day 4 after injection, the animals presented skin lesions close to the injection site so, in order to avoid future infections an antibiotic treatment was administered (Enrofloxacin, subcutaneous) for 4 days, after the treatment the wounds healed in all the animals. On day 7, the animals were treated using 3 different routes of administration: intratumoral injection, systemic injection (tail vein) and a site distant from the tumor in the upper part of the back subcutaneously. The experimental groups were the follow:
Group 1: Control, vehicle only (PBS 1×).
Group 2: empty nanoparticles (same volume as the one calculated for group 4).
Group 3: Pure growth factors 500 ng/mL (EGF:FGF, 50:50).
Group 4: Nanoparticles loaded with growth factors 100 ng/mL (total protein).

Animals corresponding to group 3 were injected with a second dose on day 22 to mimic the behavior of the GFs contained on our core-shell nanoparticles, since the availability of pure growth factors is some days but when they are delivered in the formulated system they are present in the injection site for at least 45 days (according to our previous data). Tumor size was measured, at least, every 2 days with caliper and on day 31 animals were euthanized. Tumoral volume was calculated according to The Jackson Laboratory guidelines with the following formula (Equation 1):

Volumen (mm³)=(l×w²)/2 Equation 1: Tumor volume

Where 1 is the larger side and w the shorter. Tumor size at the first measurement and the endpoint were compared for the 4 groups, values are shown in Table and Table.

TABLE 9

| | Volumen (cm³) | | | | | | | | |
|---|---|---|---|---|---|---|---|---|---|
| | Subcutaneous | | | Systemic | | | Intratumoral | | |
| | N1 | N2 | Average | N1 | N2 | Average | N1 | N2 | Average |
| Group 1 | 0.04 | 0.014 | 0.03 | 0.04 | 0.014 | 0.03 | 0.04 | 0.014 | 0.03 |
| Group 2 | 0.07 | 0.06 | 0.07 | 0.03 | 0.03 | 0.03 | 0.06 | 0.01 | 0.04 |
| Group 3 | 0.10 | 0.04 | 0.07 | 0.03 | 0.05 | 0.04 | 0.032 | 0.04 | 0.04 |
| Group 4 | 0.06 | 0.03 | 0.05 | 0.002 | 0.05 | 0.03 | 0.07 | 0.004 | 0.04 |

Tumor size at the first measurement for all treatments

TABLE 10

| | Volumen (cm³) | | | | | | | | |
|---|---|---|---|---|---|---|---|---|---|
| | Subcutaneous | | | Systemic | | | Intratumoral | | |
| | N1 | N2 | Average | N1 | N2 | Average | N1 | N2 | Average |
| Group 1 | 0.34 | 0.063 | 0.2 | 0.34 | 0.063 | 0.2 | 0.34 | 0.063 | 0.2 |
| Group 2 | 0.11 | 0.09 | 0.1 | 0.06 | 0.12 | 0.05 | 0.29 | 0.08 | 0.19 |
| Group 3 | 0.41 | 0.08 | 0.25 | 0.2 | 0.05 | 0.13 | 0.172 | 0.18 | 0.18 |
| Group 4 | 0.13 | 0.11 | 0.12 | 0.08 | 0.11 | 0.1 | 0.32 | 1.25 | 0.29 |

Tumor size day 31 for all treatments

It's important to highlight that due to the tumors had different initial volumes, just to compare tumor sizes at the endpoint could lead to conclusions that are not completely accurate since the differences at the beginning could show differences at the endpoint that are not necessarily related to the effect of the treatment. Because of this, is that the data was analyzed in two other ways, (1) difference between endpoint and initial measurement parameter that was calculated according to the equation (Equation 2):

Volume difference=V_{f}−V_{L} Equation 2: Volume difference.

Where V_{f} represents the endpoint volume and V, the volume at the first measurement. And (2) percentage of tumoral growth compared to control growth, calculated as follow (Equation 3):

$$Growth percentage = \frac{tumor size ( treatment )}{tumor size ( control )} * 100 \qquad ( 1 )$$

$$Growth percentage compared to control = \frac{growth percentage ( treatment )}{growth percentage ( control )} * 100 \qquad ( 2 )$$

$$Growth percentage compared to control . \qquad Equation 3$$

TABLE 11

| | Volumen (cm³) | | | | | | | | |
|---|---|---|---|---|---|---|---|---|---|
| | Subcutaneous | | | Systemic | | | Intratumoral | | |
| | N1 | N2 | Average | N1 | N2 | Average | N1 | N2 | Average |
| Group 1 | 0.3 | 0.049 | 0.17 | 0.3 | 0.049 | 0.17 | 0.3 | 0.049 | 0.17 |
| Group 2 | 0.04 | 0.03 | 0.04 | 0.03 | 0.09 | 0.06 | 0.23 | 0.07 | 0.15 |
| Group 3 | 0.031 | 0.04 | 0.18 | 0.17 | 0 | 0.09 | 0.14 | 0.14 | 0.14 |
| Group 4 | 0.07 | 0.08 | 0.08 | 0.08 | 0.06 | 0.07 | 0.25 | 0.246 | 0.25 |

Tumor size difference between endpoint and first measurement

TABLE 12

| | Percentage | | | | | | | | |
|---|---|---|---|---|---|---|---|---|---|
| | Subcutaneous | | | Systemic | | | Intratumoral | | |
| | N1 | N2 | Average | N1 | N2 | Average | N1 | N2 | Average |
| Group 1 | 100 | 100 | 100 | 100 | 100 | 100 | 100 | 100 | 100 |
| Group 2 | 25 | 25 | 25 | 31 | 70 | 51 | 84 | 89 | 86 |
| Group 3 | 67 | 30 | 48 | 116 | 16 | 66 | 86 | 72 | 79 |
| Group 4 | 36 | 54 | 45 | 800 | 35 | 418 | 72 | 980 | 526 |

Tumor growth percentage compared to control

When the data is analyzed as growth of the tumor in comparison to control (to eliminate the possible interference of the initial tumor size in the final volumes), it's possible to observe differences between treatments and those depend on the administration route. When the treatment is injected directly to the tumor the tumors always grew more than the control, specially the day after the injections, in this case, the increased size could also be influenced by the delay on the absorption of the substances injected directly in the tumor. By the other hand, for systemic administration of GFs tumoral growth was greater than control only the day after the injections and caused the tumors to grow more than control, different than blank NPs and NP-GFs when tumors grew more than control along all the treatment.

Finally, if the treatments are injected subcutaneously in a site distant from the tumor, none of the treatments show a tumoral growth greater than control mice (except the days immediately after the administration). Together, these results provide strong evidence that support the localized effect and continuous release of our nanoparticles, since they caused a great increase of tumoral growth when they are injected directly on the zone of the tumor where they have long stay times and reach high concentrations of GFs in the zone, but in the contrary, if they are injected subcutaneously, in a distant zone, this effect disappears, since the nanoparticles stay in the injection site avoiding the concentration of GFs in the tumor. In our model, and considering the application of this technology this data confirm that our nanoparticles shouldn't increase tumor aggressiveness on patients, since the formulation is design to be injected directly on salivary glands, in a zone distant from the tumors, feature that becomes especially relevant when reviewing the recent evidence that supports the need of develop new localized delivery systems (Varghese, 2018), so grow factors will reach concentrations high enough for salivary gland radioprotection, but not in the tumor to enhance tumor aggressiveness. Major organs and tumors were collected and fixed at the moment of euthanasia, they were prepared for histological analysis and further results.

Encapsulation effect control experiment (BioMAT'X, UAndes): Following one of the partial reports, the committee suggested to test the activity of the growth factors after the encapsulation process. Even if all the experiments performed with our formulation that have an effect, in vitro or in vivo, show that the GFs are active after it's encapsulation and release, an extra experiment was developed. 500 MC3T3-E1 cells were seeded in 24-well plates (alfa-MEM medium, 10% FBS, 1× P/S), incubated for 24 h and treated with (1) DMEM medium, (2) alfa-MEM medium, (3) NP blank and (4) NP-GFs. The cells were incubated for 5 days to allow the release of GFs and act on cells. After this time cells were fixed and stained with Alizarin Red for 45 minutes and dried at 37° C. in order to identify the mineralization degree of the cultures as an indicator of differentiation. Not all the treated cells showed the same behavior or alizarin red positive staining, but some of them had signs of calcium deposits. This slight staining and that it's only present in some of the samples could be consequence of the short incubation time, since mineralization is a sign of later stages of differentiation (14-21 days). The assays performed in this report has only a proof-of-concept objective and were selected and designed regarding the available resources, since it's a committee request was prepared with limited supplies and time. The presence of positive staining with our loaded nanocapsules suggests that the encapsulated growth factors are active since can induce osteoblastic differentiation in MC3T3-E1 cells.

Animal welfare evaluation (CemyQ, UFRO): All animal experimental procedures, as administration of drugs, new diet or supplementation evaluation, invasive surgical procedures, or any other manipulation that could induce stress, discomfort, pain or affect animal welfare, must include an “Animal Supervision Protocol”. One of the most used is the proposed by Morton and Griffiths “Guidelines on the recognition of pain, distress and discomfort in experimental animals and a hypothesis for assessment: Vet Rec 116: 431-436, 1985”, attached below. This protocol allows to quantify pain and suffering caused by a procedure in animals so it's possible to take palliative measures to provide relief or eventually to give anticipate end to the procedure, as needed. The animal supervision protocol considers 4 characteristics to evaluate in an animal, to which it's assigned a score from 0 to 3 according to the variables in the following table (Table):

TABLE 13

| Characteristic | Variable |
|---|---|
| Weightloss | Absent |
| | Less than 10% |
| | Between 10 and 20% |
| | Greater that 20% |
| Appearance | Normal |
| | Bad hair |
| | Presence of eye or nasal discharge |
| | Abnormal posture |
| Spontaneous behavior | Normal |
| | Small changes |
| | Inactivity |
| | Very restless or immobile rats |
| Behavior against | Normal |
| manipulation | Small changes |
| | Moderate changes |
| | Aggressive or comatose rats |
| Vital signs | Normal |
| | Small changes |
| | Moderate changes in heart or breathing rate |
| | Significant changes in heart or respiratory rate |

Animal welfare parameters

These parameters need to be evaluated weekly or daily by the responsible researcher, depending on the experimental protocol. If an animal gets a score above 3 in more than one variable, it will be raised (considered) to a score of 4. According to the following total score (Table), the actions that will be taken by the vet to diminish animal suffering will be stablished:

TABLE 14

| Final score | |
|---|---|
| 0-4 | Normal |
| 5-9 | Carefully supervision and analgesic administration will be |
| | evaluated |
| 10-14 | Intense suffering, analgesics are needed, and euthanasia will |
| | be evaluated |
| 15-20 | Stop procedure and euthanize |

Final score and actions

Animals were evaluated daily by work team members and CEMyQ workers. On weekends and holidays, animals were supervised and fed by the CEMyQ's bioterium worker Mr. Pedro Seguel.

Along the experiment, no pain or infections were observed, either circumstances that caused pain or suffering, so it wasn't necessary to euthanize any animal before day 30. Animals belonging to 30 days experimental group didn't show complications and food intake was relatively normal. A group of 5 animals that should be evaluated at day 90 started to present some changes from day 45, reducing food intake and behavioral changes, weakness and hypoactivity.

Considering these conditions, we determinate that the animals won't be able to tolerate irradiation therapy until day 90 and 180, so it was decided to eliminate these stages from the proposed research, keeping only the animals treated and evaluated until days 30 (definitely) and d45-60 (if possible).

At the moment of analyze the behavior of the animals corresponding to irradiated group:
The day after the irradiation, animals showed difficulty and disinterest on normal feeding, although the food was fragmented to facilitate the process. This situation was better the subsequent day, after that feeding returned to normal. Water intake was normal.
No behavioral changes were observed in the group of animals corresponding to the analysis on day 30, for example, more aggressiveness or hyperactivity.
No macroscopic morphological changes were observed in the group of animals corresponding to the analysis on day 30, as changes in size or shape.

Nevertheless, during the experiment, the animals from the irradiated group showed a decrease in the volume and amount of hair in the irradiation zone, there were even zones totally free of hair.

At the moment of euthanasia each mouse was weighed. When preliminary results were analyzed, statistical analysis showed no significative differences between compared animals (p=0.036). At the moment of euthanasia, the mean weight of the group of irradiated animals was 29.49 g, while the average of the un-irradiated group has a mean weight of 29.30 g.

### EXAMPLE 9. COMBINATION OF THE GROWTH FACTORS OFFERS ADVANTAGES OVER THE USE OF INDIVIDUAL GROWTH FACTORS

The composition of the invention comprising a release-controlled nanocarrier consisting of core-shell nanoparticles wherein the nanoparticles comprises EGF growth factor and bFGF growth factor, gave rise to the best results in the prevention of radiation-induced damage, such as it is demonstrated below.

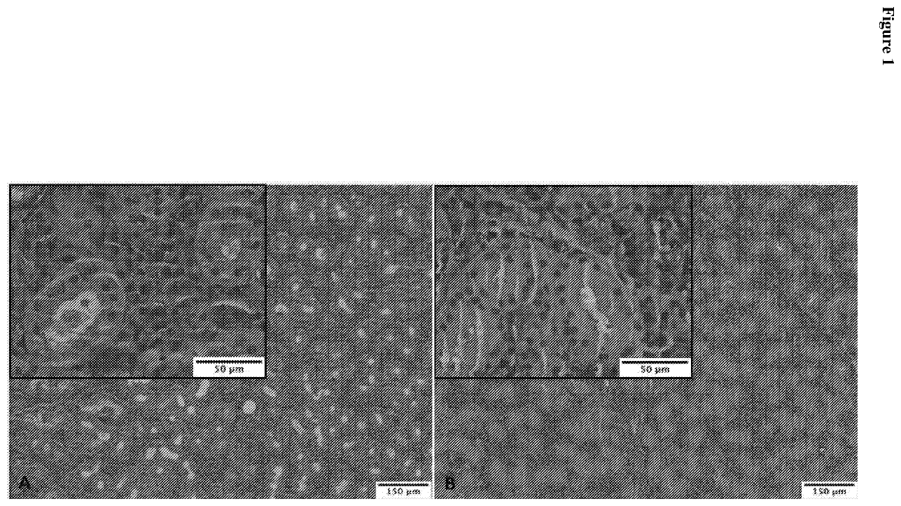

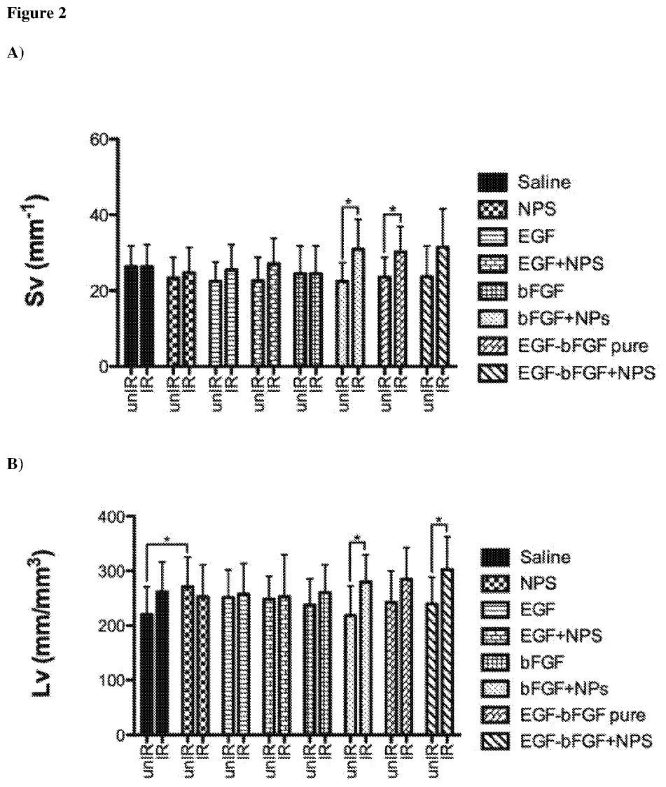

Such as it is shown in FIG. 1, wherein when glands from the un-irradiated groups (controls) are compared to glands in irradiated groups (experimental) treated with the composition of the invention, no significant histological and stereological differences were observed neither in the secretory portion nor in the stromal connective tissue, demonstrating the protective effect of the composition of the invention. This is further confirmed by FIG. 2 wherein it is shown that the excretory portion (critical for salivation) showed significative (quantitative) differences, rendering the positive effect of individual as well as dual growth factor therapy, with superiority for the composition of the invention comprising dual growth factor (EGF+bFGF+NPs). Indeed, the IR group receiving the composition of the invention (EGF+bFGF+NPs), showed values significantly higher for Lv and Sv in ducts than in the unIR groups (and higher but less for individual growth factor-IR groups). Basically, this indicates an increase in length and surface per cubic millimetre of the injured ducts in these groups, preferably when treated with the composition of the invention comprising release-controlled nanocarrier consisting of core-shell nanoparticles which in turn comprises EGF growth factor and bFGF growth factor.

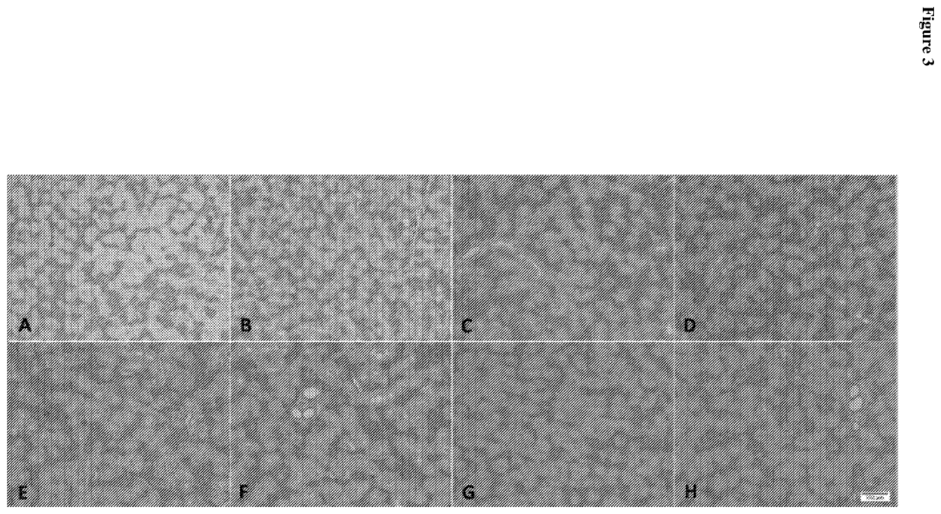

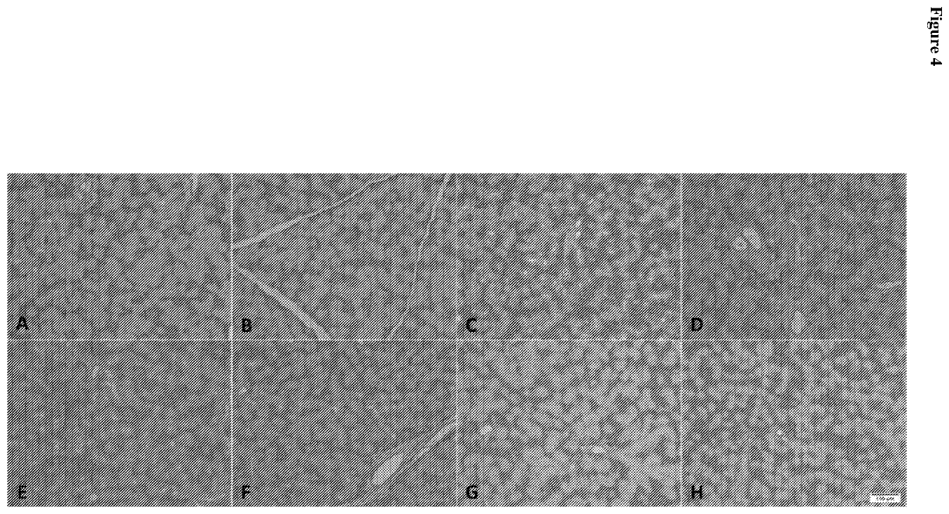

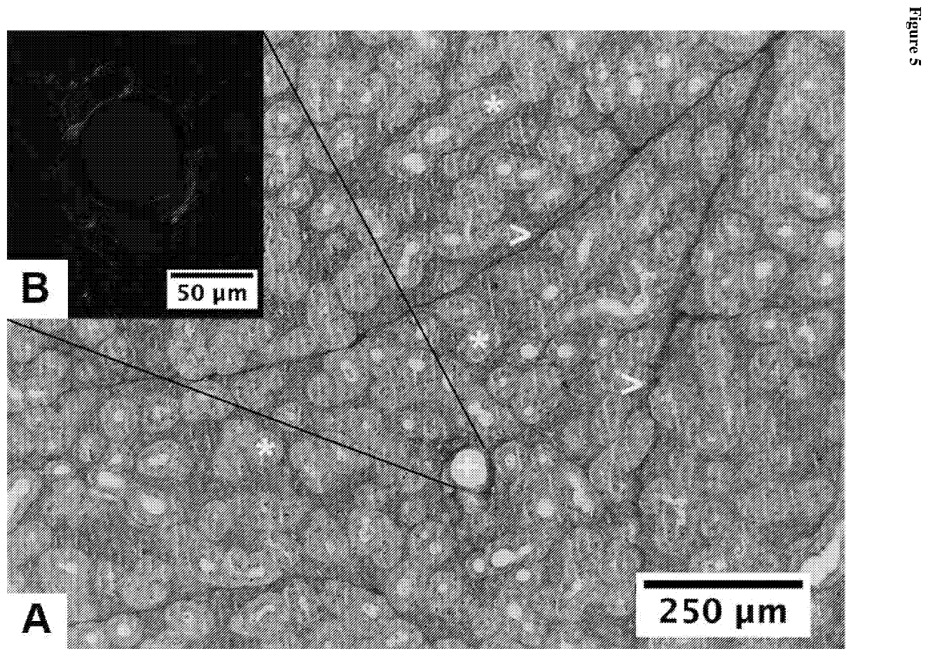

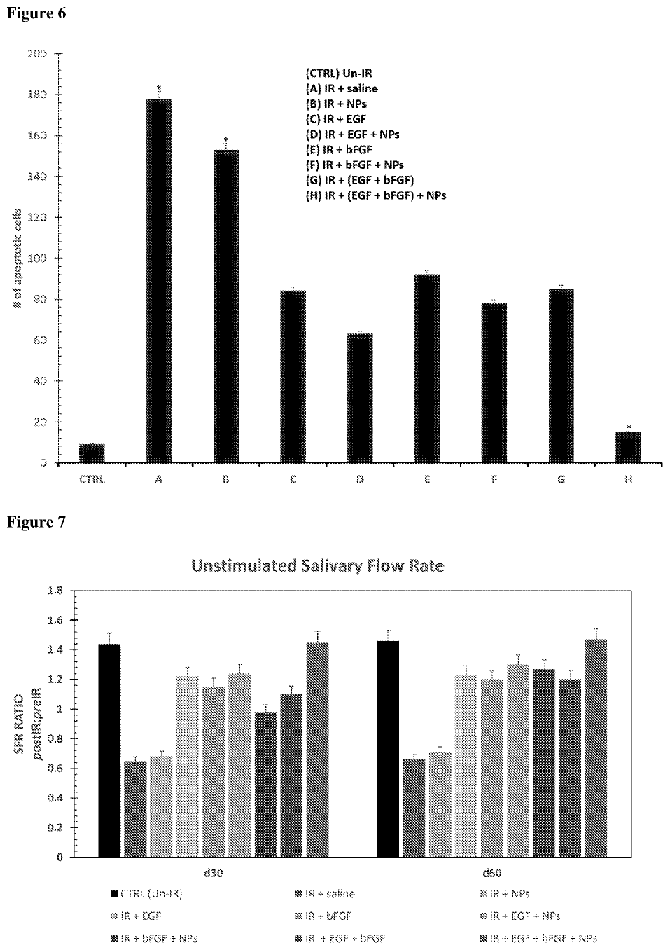

The positive results obtained with the composition of the invention is further confirmed by FIG. 3 and FIG. 4 wherein glands of Un-IR (FIG. 3) and IR (FIG. 4) groups were compared and analyzed stereologically (saline injection for syringe effect vs blank NPs vs Pure individual GFs vs

Pure cocktail GFs vs individual GF NPs vs cocktail GF NPs), it was possible to observe some key differences, where adenomeres and granular contoured ducts showed signs of hypertrophy and slight hyperplasia. Staining with PAS and AA at pH 2.5 showed a “higher secretion from glandular adenomeres” in all the groups with growth factors; mild in individual GF groups and moderate in cocktail GF groups, hence, once again demonstrating the beneficial effect of the composition of the invention in irradiated glands.

Moreover, such as it is shown in FIG. 7, wherein salivary function assessment determined at day 30 (d30) and day 60 (d60) post-IR, expressed below as ratio of post-IR SFR (salivary flow rate) to pre-IR SFR (mean±standard error), no significant differences were detected between control (un-IR) and experimental irradiated mice which were treated with the composition of the invention.

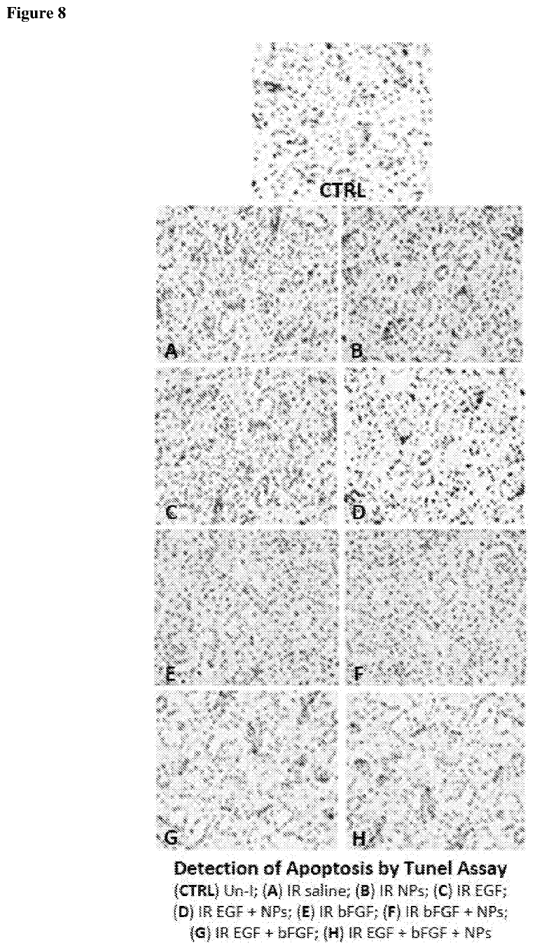

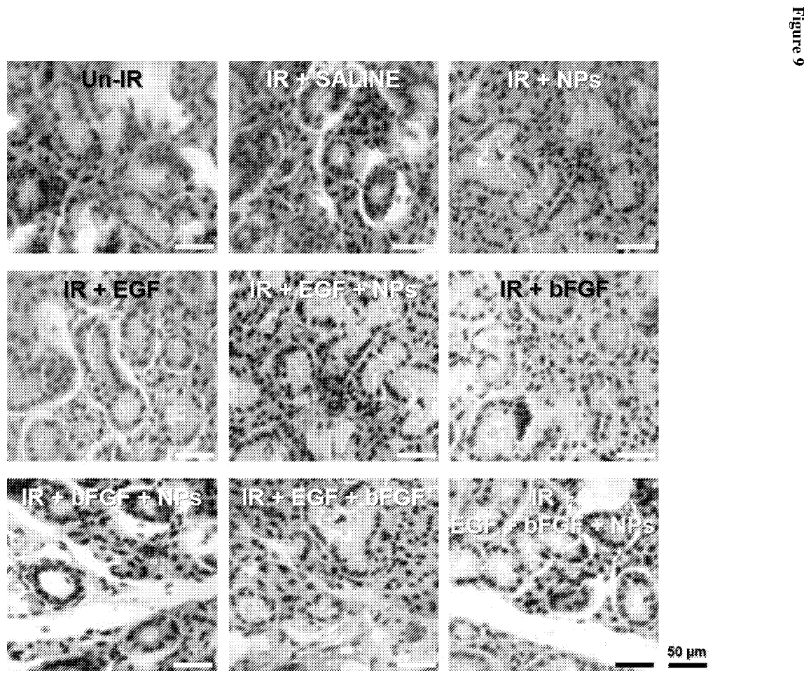

Moreover, FIG. 8 also confirms the positive results obtained with the composition of the invention because the lowest effect on apoptosis was shown when the composition of the invention was used in irradiated samples.

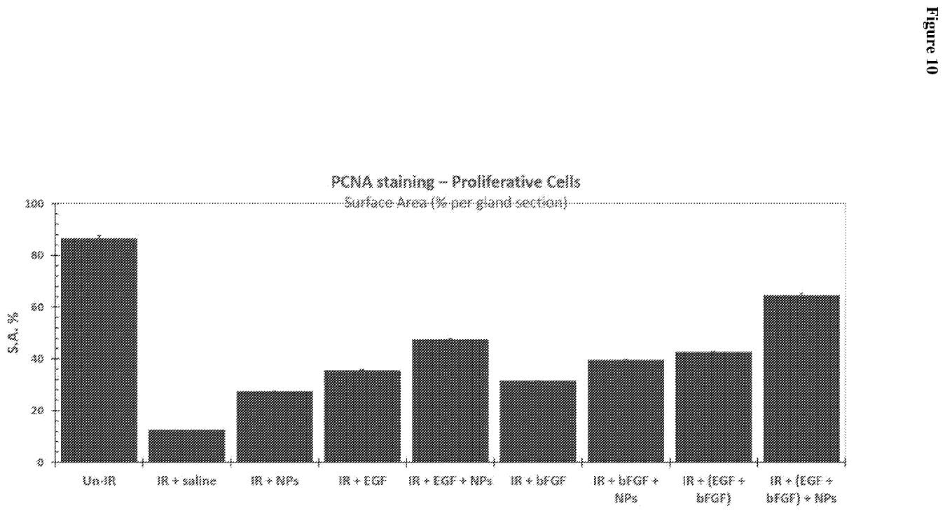

Finally, the number of proliferative cells (acinar and granular convoluted tubular cells) were determined in differ conditions such as it is shown in FIG. 9 and FIG. 10. The number of proliferative cells significantly increased in all experimental groups when compared to control (unirradiated). Of note, again, the highest number of proliferative cells was obtained when the composition of the invention was used, which in fact shows the lowest effect in apoptosis as described above.

Consequently, these findings confirm the protective and regenerative effect of the composition of the invention: (EGF+bFGF)+NPs strategy, over-time, with a favorable capacity to modulate effects via customizing the core-shell format, growth factor load and pharmacokinetic profile.

1. Pharmaceutical composition comprising a release-controlled nanocarrier consisting of core-shell nanoparticles, wherein the nanoparticles comprises EGF growth factor and bFGF growth factor.

2. Pharmaceutical composition, according to claim 1, comprising a release-controlled nanocarrier consisting of core-shell nanoparticles wherein the nanoparticle core comprises EGF growth factor and the nanoparticle shell is construed via electrostatic-based self-assembly of natural polymers and comprises bFGF growth factor.

3. Pharmaceutical composition, according to any of the previous claims, wherein the core-shell nanoparticles comprises a lipid core and the nanoparticle shell comprises a plurality of layers made of natural polymers.

4. Pharmaceutical composition, according to any of the previous claims, wherein the natural polymers are pre-selected blends from: alginates, chitins, chitosans, cellulose-derivates, gelatins and/or hyaluronans.

5. Pharmaceutical composition, according to any of the previous claims, wherein the lipid core comprises liposomes or solid lipid nanoparticles.

6. Pharmaceutical composition, according to any of the claims 1 to 5, for use in preventing radiation-induced damage in cancer patients treated with radiotherapy.

7. Pharmaceutical composition for use, according to claim 6, in preventing radiation-induced damage in patients suffering from head and neck cancer who are treated with radiotherapy.

8. Pharmaceutical composition for use, according to any of the claims 5 to 7, in preventing salivary glands damage and/or dry mouth complications.

9. Pharmaceutical composition for use, according to any of the claims 5 to 8, in preventing xerostomia, halitosis, burning mouth syndrome or ulcers.

10. Pharmaceutical composition for use, according to any of the claims 5 to 9, in the reparation, reconstruction or regeneration of the salivary glands.

11. Pharmaceutical composition for use, according to any of the claims 5 to 10, wherein the growth factors are continuously delivered for a pre-established period of time, preferably for 30-45 days.

12. Pharmaceutical composition for use, according to any of the claims 5 to 11, wherein the growth factors are sequentially delivered in a manner that bFGF is released fist and EGF is delayed for a pre-established period of time.

13. Pharmaceutical composition for use, according to any of the claims 5 to 12, wherein the growth factors are delivered at a pre-established rate.

14. Pharmaceutical composition for use, according to any of the claims 5 to 13, wherein the composition is directly injected into the high-risk/susceptible site, preferably into the salivary glands.

15. Pharmaceutical composition for use, according to any of the claims 5 to 14, wherein 100-500 ng/mL are administered 24 hours to 6 hours before radiotherapy session.
